(19) United States
(12) Patent Application Publication
(10) Pub. No.: US 2017/0143738 A1
(43) Pub. Date: May 25, 2017

(54) SOLID FORMULATION AND METHOD FOR STABILIZING THE SAME

(71) Applicant: EA Pharma Co., Ltd., Tokyo (JP)

(72) Inventors: Takahiko Ando, Kawasaki-shi, Kanagawa (JP); Hirokazu Hagio, Kawasaki-shi, Kanagawa (JP); Takashi Matsushita, Kawasaki-shi, Kanagawa (JP); Yusuke Ito, Kawasaki-shi, Kanagawa (JP); Makoto Sugiura, Kawasaki-shi, Kanagawa (JP)

(21) Appl. No.: 15/320,651

(22) PCT Filed: Jun. 24, 2015
(86) PCT No.: PCT/JP2015/068240

§ 371 Date: Dec. 20, 2016

(30) Foreign Application Priority Data
Jun. 25, 2014 (JP) 2014-130091

(51) Int. Cl. A61K 31/554; A61K 9/20; A61K 9/28

(52) U.S. Cl. CPC A61K 31/554; A61K 9/2866; A61K 9/2009; A61K 9/2068; A61K 9/2031; A61K 9/2813

(57) ABSTRACT

The present invention relates to stabilization of a certain benzothia(dia)zepine derivative in a solid formulation containing the same. In the present invention, in a solid formulation containing (A) the benzothia(dia) zepine derivative mentioned above and (B) a specific plasticizer, the aforementioned ingredient (A) is isolated from the aforementioned ingredient (B), or alternatively, in the case of the aforementioned ingredient (A) not being isolated from the aforementioned ingredient (B), an amount of the aforementioned ingredient (B) is controlled to 0.9% by weight or less based on the total weight of the solid formulation, or the solid formulation is provided with a form containing at least one core and at least one coating layer or capsule layer enclosing at least a part of the core, the aforementioned ingredient (A) is mixed into the core, the aforementioned ingredient (B) is mixed into the coating layer or capsule layer, and an amount of the aforementioned ingredient (B) in the coating layer or capsule layer is controlled to 45% by weight or less based on the total weight of the coating layer or capsule layer.

### TECHNICAL FIELD

The present invention relates to stabilization of a solid formulation containing a benzothia(dia)zepine derivative or a pharmaceutically acceptable salt, solvate, or solvate of such a salt.

### BACKGROUND ART

It is known that some benzothia(dia)zepine derivatives function as inhibitors of IBAT (Ileal Bile Acid Transporter) (Patent Document 1). The inhibitors of IBAT are useful in the treatment of dyslipidemic conditions and disorders such as hyperlipidemia, hypertrigliceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL).

In addition, the benzothia(dia)zepine derivatives mentioned above are also useful in the treatment of functional constipation and constipation-dominant irritable bowel syndrome (C-IBS) (Patent Document 2 and Patent Document 3).

### PRIOR ART DOCUMENTS

### Patent Documents

Patent Document 1: Japanese Patent No. 3665055

Patent Document 2: Japanese Patent No. 4870552

Patent Document 3: Japanese Patent No. 5421326

### DISCLOSURE OF INVENTION

### Technical Problems

The aforementioned benzothia(dia)zepine derivatives or pharmaceutically acceptable salts, solvates, or solvates of such salts (hereinafter, simply referred to as “benzothia(dia)zepine derivative” in some cases) are stable compounds per se. For example, they are stable over time even under an atmosphere of high temperature and/or high humidity.

However, it has been found that there is a problem in that in the case of blending the aforementioned benzothia(dia)zepine derivative in a solid formulation, the benzothia(dia)zepine derivative may become unstable and be decomposed over time. In particular, related substances derived from decomposition of the aforementioned benzothia(dia)zepine derivative in a solid formulation may be produced under open atmosphere.

The present invention has an object to stabilize certain benzothia(dia)zepine derivatives in solid formulations containing the same, and provide a solid formulation containing the stabilized derivative mentioned above.

### Technical Solution

The object of the present invention can be achieved, in a solid formulation containing (A) a certain benzothia(dia)zepine derivative and (B) a specified plasticizer, by
isolating the above-mentioned ingredient (A) from the above-mentioned ingredient (B);
   or alternatively
in the case of the above-mentioned ingredient (A) not being isolated from the above-mentioned ingredient (B),
controlling an amount of the above-mentioned ingredient (B) to 0.9% by weight or less based on the total weight of the solid formulation,
or
providing the solid formulation with a form containing at least one core and at least one coating layer or capsule layer enclosing at least a part of the core, mixing the above-mentioned ingredient (A) into the core, mixing the above-mentioned ingredient (B) into the coating layer or capsule layer, and controlling an amount of the above-mentioned ingredient (B) in the coating layer or capsule layer to 45% by weight or less based on the total weight of the coating layer or capsule layer.

In the present specification, the term “weight” has the same meaning as that of “mass”. Therefore, “% by weight” and “part (s) by weight” have the same meanings as those of “% by mass” and “part(s) by mass”, respectively.

The first aspect of the present invention relates to a solid formulation characterized by containing:
(A) a compound of formula (I) or (I′):

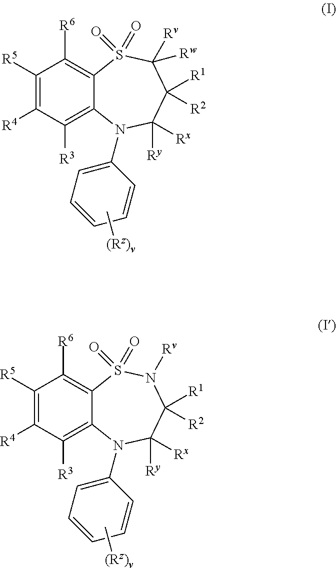

wherein:
R^{v} and R^{w} are independently selected from hydrogen or C₁₋₆ alkyl;
R¹ and R² are independently selected from C₁₋₆ alkyl;
R^{x} and R^{y} are independently selected from hydrogen or C₁₋₆ alkyl, or one of R^{x} and R^{y} is hydrogen or C₁₋₆ alkyl and the other is hydroxy or C₁₋₆ alkoxy;
R^{z} is selected from halo, nitro, cyano, hydroxy, amino, carboxy, carbamoyl, mercapto, sulphamoyl, C₁₋₆ alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl) carbamoyl, N,N—(C₁₋₆ alkyl)₂ carbamoyl, C₁₋₆ alkyl S(O)ₐ wherein a is 0 to 2, C₁₋₆ alkoxycarbonyl, C₁₋₆ alkoxycarbonylamino, ureido, N′—(C₁₋₆ alkyl)ureido, N—(C₁₋₆ alkyl)ureido, N′,N′—(C₁₋₆ alkyl)₂ ureido, N′—(C₁₋₆ alkyl)-N—(C₁₋₆ alkyl)ureido, N′,N′—(C₁₋₆ alkyl)₂-N—(C₁₋₆ alkyl) ureido, N—(C₁₋₆ alkyl) sulphamoyl and N,N—(C₁₋₆ alkyl)₂ sulphamoyl;
v is 0 to 5;
one of R⁴ and R⁵ is a group of formula (IA):

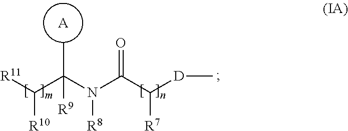

R³ and R⁶, and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆ alkyl and b is 0 to 2;
Ring A is aryl or heteroaryl; wherein Ring A is optionally substituted by one or more substituents selected from R¹⁷;
R⁷ is hydrogen, C₁₋₄ alkyl, carbocyclyl or heterocyclyl;
wherein R⁷ is optionally substituted by one or more substituents selected from R¹⁸;
R⁸ is hydrogen or C₁₋₄ alkyl;
R⁹ is hydrogen or C₁₋₄ alkyl;
R¹⁰ is hydrogen, C₁₋₄ alkyl, carbocyclyl or heterocyclyl; wherein R¹⁰ is optionally substituted by one or more substituents selected from R¹⁹;
R¹¹ is carboxy, sulpho, sulphino, phosphono, tetrazolyl, —P(O)(OR^{c})(OR^{d}), —P(O)(OH)(OR^{e}), —P(O)(OH)(R^{d}) or
—P(O)(OR^{c})(R^{d}) wherein R^{c} and R^{d} are independently selected from C₁₋₆ alkyl; or R¹¹ is a group of formula (IB):

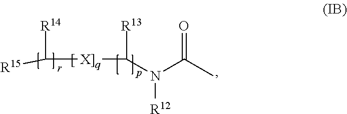

wherein:
X is —N(R^{q})—, —N(R^{q})C(O)—, —O—, or —S(O)ₐ—; wherein a is 0 to 2 and R^{q} is hydrogen or C₁₋₄ alkyl;
R¹² is hydrogen or C₁₋₄ alkyl;
R¹³ and R¹⁴ are independently selected from hydrogen, C₁₋₄ alkyl, carbocyclyl, heterocyclyl or R²³; wherein said C₁₋₄ alkyl, carbocyclyl or heterocyclyl may be independently optionally substituted by one or more substituents selected from R²⁰;
R¹⁵ is carboxy, sulpho, sulphino, phosphono, tetrazolyl, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆ alkyl; or R¹⁵ is a group of formula (IC):

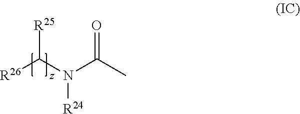

wherein:
R²⁴ is selected from hydrogen or C₁₋₄ alkyl;
R²⁵ is selected from hydrogen, C₁₋₄ alkyl, carbocyclyl, heterocyclyl or R²⁷; wherein said C₁₋₄ alkyl, carbocyclyl or heterocyclyl may be independently optionally substituted by one or more substituents selected from R²⁸;
R²⁶ is selected from carboxy, sulpho, sulphino, phosphono, tetrazolyl, —P(O)(OR^{g})(OR^{h}), —P(O)(OH)(OR^{g}), —P(O)(OH)(R^{g}) or —P(O)(OR^{g})(R^{h}) wherein R^{g} and R^{h} are independently selected from C₁₋₆ alkyl;
p is 1-3; wherein the meanings of R¹³ may be the same or different;
q is 0-1;
r is 0-3; wherein the meanings of R¹⁴ may be the same or different;
m is 0-2; wherein the meanings of R¹⁰ may be the same or different;
n is 1-3; wherein the meanings of R⁷ may be the same or different;
z is 0-3; wherein the meanings of R²⁵ may be the same or different;
R¹⁶, R¹⁷ and R¹⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl) amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl) sulphamoyl and N,N—(C₁₋₄ alkyl)₂ sulphamoyl; wherein R¹⁶, R¹⁷ and R¹⁸ may be independently optionally substituted on carbon by one or more R²¹;
R¹⁹, R²⁰, R²³, R²⁷ and R²⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆ alkyl; wherein R¹⁹, R²⁰, R²³, R²⁷ and R²⁸ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl, or a pharmaceutically acceptable salt, solvate, or solvate of such a salt; and
(B) at least one selected from the group consisting of polyethylene glycol, propylene glycol, glycerol, glyceryl triacetate, triethyl acetylcitrate, dibutyl sebacate, diethyl phthalate, castor oil, a copolymer of propylene oxide and ethylene oxide, triacetin, triethyl citrate, and a mixture thereof,
   wherein
   the above-mentioned ingredient (A) is isolated from the above-mentioned ingredient (B), or alternatively in the case of the above-mentioned ingredient (A) not being isolated from the above-mentioned ingredient (B),
   an amount of the above-mentioned ingredient (B) is 0.9% by weight or less based on the total weight of the solid formulation,
      or
   the solid formulation contains
   at least one core, and
   at least one coating layer or capsule layer enclosing at least a part of the above-mentioned core,
   where
   the core contains the above-mentioned ingredient (A), the coating layer or capsule layer contains the above-mentioned ingredient (B), and
   an amount of the above-mentioned ingredient (B) in the coating layer or capsule layer is 45% by weight or less based on the total weight of the coating layer or capsule layer.

The first aspect is preferably a solid formulation which contains:

at least one core, and
at least one coating layer or capsule layer enclosing at least a part of the core,
where
the core contains the above-mentioned ingredient (A),
the coating layer or capsule layer contains the above-mentioned ingredient (B), and
at least one isolation layer is provided between the core and the coating layer or capsule layer, or alternatively in the case of the core contacting the coating layer or capsule layer,
an amount of the above-mentioned ingredient (B) in the coating layer or capsule layer is 0.9% by weight or less based on the total weight of the solid formulation,
   or
an amount of the above-mentioned ingredient (B) in the coating layer or capsule layer is 45% by weight or less based on the total weight of the coating layer or capsule layer.

In the case of the core contacting the coating layer or capsule layer, an amount of the above-mentioned ingredient (B) in the coating layer or capsule layer preferably ranges from 0.1 to less than 40% by weight based on the total weight of the coating layer or capsule layer.

The coating layer or capsule layer can be present in a ratio ranging from 1 to 20% by weight based on the total weight of the solid formulation.

The coating layer or capsule layer preferably further contains at least one selected from the group consisting of a water-soluble polymer other than polyethylene glycol, a colorant, a lubricant, and wax.

The water-soluble polymer is preferably hydroxypropyl methylcellulose.

The colorant is preferably selected from the group consisting of titanium oxide, iron oxide, zinc oxide, tar pigment, and lake pigment.

The lubricant is preferably talc.

The wax is preferably carnauba wax.

The core preferably contains at least one additive selected from the group consisting of a filler, a disintegrant, a binder, a lubricant, and a fluidizer.

The solid formulation according to the present invention is preferably a film-coated tablet or a capsule.

The amount of the above-mentioned ingredient (B) may range from 0.1 to 20% by weight based on the total weight of the above-mentioned ingredient (A).

The above-mentioned ingredient (A) is preferably selected from the group consisting of:
1,1-dioxo-3,3-dibutyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,2,5-benzothiadiazepin e;
1,1-dioxo-3,3-dibutyl-5-phenyl-7-methylthio-8-(N—{(R)-α-[N—((S)-1-carboxy-2-(R)-hydroxypropyl)carbamoyl]-4-hydroxy benzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl)carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiazepine.

The above-mentioned ingredient (A) is more preferably 1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiazepine, that is, Elobixibat.

The amount of the above-mentioned ingredient (A) may range from 0.01 to 50% by weight based on the total weight of the solid formulation.

The amount of the above-mentioned ingredient (A) may range from 1 to 20 mg.

The above-mentioned polyethylene glycol has an average molecular weight preferably ranging from 200 to 20,000.

The solid formulation according to the present invention is preferably in the form of a tablet having a diameter ranging from 5 to 11 mm.

The solid formulation according to the present invention is preferably intended for treating or preventing constipation in a warm-blooded animal including a human being. The above-mentioned constipation may be functional constipation or constipation-predominant irritable bowel syndrome.

The second aspect of the present invention relates to a method for stabilizing the following ingredient (A) in a solid formulation containing:

(A) a compound represented by the above-mentioned formula (I) or (I′), or a pharmaceutically acceptable salt, solvate, or solvate of such a salt; and
(B) at least one selected from the group consisting of polyethylene glycol, propylene glycol, glycerol, glyceryl triacetate, triethyl acetylcitrate, dibutyl sebacate, diethyl phthalate, castor oil, a copolymer of propylene oxide and ethylene oxide, triacetin, triethyl citrate, and a mixture thereof,
the method being characterized by including:
isolating the above-mentioned ingredient (A) from the above-mentioned ingredient (B); or alternatively in the case of the above-mentioned ingredient (A) not being isolated from the above-mentioned ingredient (B),
controlling an amount of the above-mentioned ingredient (B) to 0.9% by weight or less based on the total weight of the solid formulation,
   or
providing the solid formulation with a form containing at least one core and at least one coating layer or capsule layer enclosing at least a part of the core,
mixing the above-mentioned ingredient (A) into the core, mixing the above-mentioned ingredient (B) into the coating layer or capsule layer, and
controlling an amount of the above-mentioned ingredient (B) in the coating layer or capsule layer to 45% by weight or less based on the total weight of the coating layer or capsule layer.

### Effects of the Invention

In accordance with the present invention, a certain benzothia(dia)zepine derivative in a solid formulation containing the same can be stabilized, and a solid formulation containing the stabilized derivative can be provided.

The benzothia(dia)zepine derivatives in the solid formulations according to the present invention are stable over time even under an atmosphere at high temperature and/or in high moisture. Therefore, even if the solid formulations according to the present invention are allowed to stand under an atmosphere at high temperature and/or in high moisture, production of related substance(s) derived due to decomposition of the benzothia(dia)zepine derivatives mentioned above can be prevented or reduced. In particular, the solid formulations of the present invention are stable under open atmosphere.

Therefore, the solid formulations of the present invention can be stored for a long period of time, and the pharmaceutical effects of the benzothia(dia)zepine derivatives contained in the solid formulations can be maintained. In addition, in the solid formulations according to the present invention, coloration of the above-mentioned formulations caused by related substance(s) derived from decomposition of the benzothia(dia)zepine derivatives mentioned above can be prevented or reduced. In particular, the solid formulations of the present invention can be stable even under an atmosphere at high temperature and/or in high moisture in the summer season.

### MODE FOR CARRYING OUT THE INVENTION

As a result of diligent studies of the cause of non-stabilization of the above-mentioned benzothia(dia)zepine derivatives (in particular, production of related substance(s) thereof) in solid formulations containing the derivatives, the inventors of the present application determined the cause. That is, the above-mentioned benzothia(dia)zepine derivatives contact a relatively large amount of specific plasticizer(s) in solid formulations containing the same, thus causing unstabilization of the derivatives.

In the present invention, in a solid formulation containing the above-mentioned benzothia(dia)zepine derivative and the above-mentioned specific plasticizer, isolating the above-mentioned two ingredients; or alternatively, in the case of the above-mentioned one ingredient not being isolated from the other ingredient, controlling an amount of the above-mentioned plasticizer to 0.9% by weight or less based on the total weight of the solid formulation; or providing the solid formulation with a form containing at least one core and at least one coating layer or capsule layer enclosing at least a part of the core, mixing the above-mentioned ingredient (A) into the core, mixing the above-mentioned ingredient (B) into the coating layer or capsule layer, and controlling an amount of the above-mentioned ingredient (B) in the coating layer or capsule layer to 45% by weight or less based on the total weight of the coating layer or capsule layer are performed. Thereby, production of the related substance(s) derived due to unstabilization of the above-mentioned benzothia(dia)zepine derivative can be prevented or reduced.

Hereinafter, the embodiments for carrying out the present invention are described in detail.

A first aspect of the present invention relates to a solid formulation containing a certain benzothia(dia)zepine derivative and a specific plasticizer, wherein the benzothia(dia)zepine derivative is isolated from the plasticizer; or alternatively, in the case of the above-mentioned benzothia(dia)zepine derivative not being isolated from the plasticizer, an amount of the above-mentioned plasticizer is 0.9% by weight or less based on the total weight of the solid formulation; or the solid formulation is equipped with at least one core and at least one coating layer or capsule layer enclosing at least a part of the core, where the core contains the above-mentioned ingredient (A), the coating layer or capsule layer contains the above-mentioned ingredient (B), and an amount of the above-mentioned ingredient (B) in the coating layer or capsule layer is 45% by weight or less based on the total weight of the coating layer or capsule layer.

The benzothia(dia)zepine derivatives usable in the present invention are preferably (A) compounds represented by the following formula (I) or (I′):

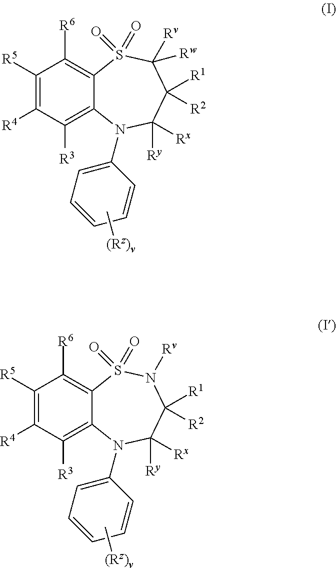

wherein:
R^{v} and R^{w} are independently selected from hydrogen or C₁₋₆ alkyl;
R¹ and R² are independently selected from C₁₋₆ alkyl;
R^{x} and R^{y} are independently selected from hydrogen or C₁₋₆ alkyl, or one of R^{x} and R^{y} is hydrogen or C₁₋₆ alkyl and the other is hydroxy or C₁₋₆ alkoxy;
R^{z} is selected from halo, nitro, cyano, hydroxy, amino, carboxy, carbamoyl, mercapto, sulphamoyl, C₁₋₆ alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl) carbamoyl, N,N—(C₁₋₆ alkyl)₂ carbamoyl, C₁₋₆ alkyl S(O)ₐ wherein a is 0 to 2, C₁₋₆ alkoxycarbonyl, C₁₋₆ alkoxycarbonylamino, ureido, N′—(C₁₋₆ alkyl)ureido, N—(C₁₋₆ alkyl)ureido, N′,N′—(C₁₋₆ alkyl)₂ ureido, N′—(C₁₋₆ alkyl)-N—(C₁₋₆ alkyl)ureido, N′,N′—(C₁₋₆ alkyl)₂-N—(C₁₋₆ alkyl) ureido, N—(C₁₋₆ alkyl) sulphamoyl and N,N—(C₁₋₆ alkyl)₂ sulphamoyl;
v is 0 to 5;
one of R⁴ and R⁵ is a group of formula (IA):

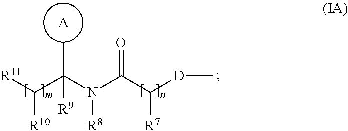

R³ and R⁶, and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆ alkyl and b is 0 to 2;
Ring A is aryl or heteroaryl; wherein Ring A is optionally substituted by one or more substituents selected from R¹⁷;
R⁷ is hydrogen, C₁₋₄ alkyl, carbocyclyl or heterocyclyl; wherein R⁷ is optionally substituted by one or more substituents selected from R¹⁸;
R⁸ is hydrogen or C₁₋₄ alkyl;
R⁹ is hydrogen or C₁₋₄ alkyl;
R¹⁰ is hydrogen, C₁₋₄ alkyl, carbocyclyl or heterocyclyl; wherein R¹⁰ is optionally substituted by one or more substituents selected from R¹⁹;
R¹¹ is carboxy, sulpho, sulphino, phosphono, tetrazolyl, —P(O)(OR^{c})(OR^{d}), —P(O)(OH)(OR^{e}), —P(O)(OH)(R^{d}) or —P(O)(OR^{c})(R^{d}) wherein R^{c} and R^{d} are independently selected from C₁₋₆ alkyl; or R¹¹ is a group of formula (IB):

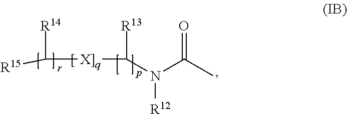

wherein:
X is —N(R^{q})—, —N(R^{q})C(O)—, —O—, or —S(O)ₐ—; wherein a is 0 to 2 and R^{q} is hydrogen or C₁₋₄ alkyl;
R¹² is hydrogen or C₁₋₄ alkyl;
R¹³ and R¹⁴ are independently selected from hydrogen, C₁₋₄ alkyl, carbocyclyl, heterocyclyl and R²³; wherein said C₁₋₄ alkyl, carbocyclyl or heterocyclyl may be independently optionally substituted by one or more substituents selected from R²⁰;
R¹⁵ is carboxy, sulpho, sulphino, phosphono, tetrazolyl, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆ alkyl; or R¹⁵ is a group of formula (IC):

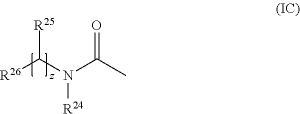

wherein:
R²⁴ is selected from hydrogen and C₁₋₄ alkyl;
R²⁵ is selected from hydrogen, C₁₋₄ alkyl, carbocyclyl, heterocyclyl and R²⁷; wherein said C₁₋₄ alkyl, carbocyclyl or heterocyclyl may be independently optionally substituted by one or more substituents selected from R²⁸;
R²⁶ is selected from carboxy, sulpho, sulphino, phosphono, tetrazolyl, —P(O)(OR^{g})(OR^{h}), —P(O)(OH)(OR^{g}), —P(O)(OH)(R^{g}) or —P(O)(OR^{g})(R^{h}) wherein R^{g} and R^{h} are independently selected from C₁₋₆ alkyl;
p is 1-3; wherein the meanings of R¹³ may be the same or different;
q is 0-1;
r is 0-3; wherein the meanings of R¹⁴ may be the same or different;
m is 0-2; wherein the meanings of R¹⁰ may be the same or different;
n is 1-3; wherein the meanings of R⁷ may be the same or different;
z is 0-3; wherein the meanings of R²⁵ may be the same or different;
R¹⁶, R¹⁷ and R¹⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl) amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl) sulphamoyl and N,N—(C₁₋₄ alkyl)₂ sulphamoyl; wherein R¹⁶, R¹⁷ and R¹⁸ may be independently optionally substituted on carbon by one or more R²¹;
R¹⁹, R²⁰, R²³, R²⁷ and R²⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆ alkyl; wherein R¹⁹, R²⁰, R²³, R²⁷ and R²⁸ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl,
   or a pharmaceutically acceptable salt, solvate, or solvate of such a salt; or alternatively a prodrug of the same (hereinafter, simply referred to as “ingredient (A)” in some cases).

The compound of the above-mentioned formula (I) is preferably a compound represented by the following formula (I-1):

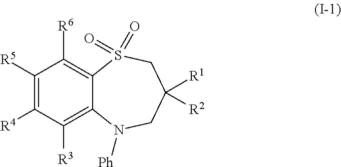

wherein
R¹ and R² are independently selected from C₁₋₆ alkyl; one of R⁴ and R⁵ is a group of the following formula (I-1A′):

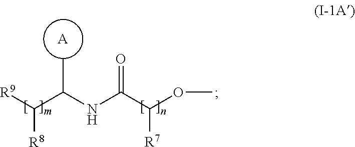

R³ and R⁶, and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl) sulphamoyl and N,N—(C₁₋₄ alkyl)₂ sulphamoyl; wherein R³ and R⁶ and the other of R⁴ and R⁵ may be optionally substituted on carbon by one or more R¹²;
Ring A is aryl or heteroaryl; wherein Ring A is optionally substituted by one or more substituents selected from R¹³;
R⁷ is hydrogen, C₁₋₄ alkyl, carbocyclyl or heterocyclyl; wherein
R⁷ is optionally substituted by one or more substituents selected from R¹⁴;
R⁸ is hydrogen, C₁₋₄ alkyl, carbocyclyl or heterocyclyl; wherein
R⁸ is optionally substituted by one or more substituents selected from R¹⁵;
R⁹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆ alkyl; or R⁹ is a group of formula (I-1B′)

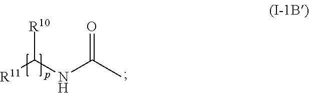

wherein:
R¹⁰ is selected from hydrogen, C₁₋₄ alkyl, carbocyclyl or heterocyclyl; wherein R¹⁰ may be independently optionally substituted by one or more substituents selected from R¹⁶;
R¹¹ is selected from carboxy, sulpho, sulphino, phosphono, —P(O)(OR^{e})(OR^{f}), —P(O)(OH)(OR^{e}), —P(O)(OH)(R^{e}) and —P(O)(OR^{e})(R^{f}) wherein R^{e} and R^{f} are independently selected from C₁₋₆ alkyl;
p is 1-3; wherein the meanings of R¹⁰ may be the same or different;
m is 0-2; wherein the meanings of R⁸ may be the same or different;
n is 1-3; wherein the meanings of R⁷ may be the same or different;
R¹², R¹³ and R¹⁴ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl) amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl;
wherein R¹², R¹³ and R¹⁴ may be independently optionally substituted on carbon by one or more R¹⁷;
R¹⁵ and R¹⁶ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, sulpho, sulphino, amidino, phosphono, —P(O)(OR^{a})(OR^{b}), —P(O)(OH)(OR^{a}), —P(O)(OH)(R^{a}) and —P(O)(OR^{a})(R^{b}), wherein R^{a} and R^{b} are independently selected from C₁₋₆ alkyl; wherein R¹⁵ and R¹⁶ may be independently optionally substituted on carbon by one or more R¹⁸;
R¹⁷ and R¹⁸ are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl.

The compound of the above-mentioned formula (I) is more preferably a compound represented by the following formula (I-2):

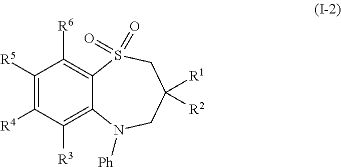

wherein
R¹ and R² are independently selected from C₁₋₆ alkyl;
one of R⁴ and R⁵ is a group of the following formula (I-2A″):

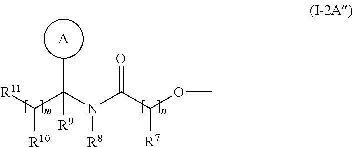

R³ and R⁶, and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl) sulphamoyl and N,N—(C₁₋₄ alkyl)₂ sulphamoyl; wherein R³ and R⁶ and the other of R⁴ and R⁵ may be optionally substituted on carbon by one or more R¹⁶;
Ring A is aryl or heteroaryl; wherein Ring A is optionally substituted by one or more substituents selected from R¹⁷;
R⁷ is hydrogen, C₁₋₄ alkyl, carbocyclyl or heterocyclyl; wherein
R⁷ is optionally substituted by one or more substituents selected from R¹⁸;
R⁸ is hydrogen or C₁₋₄ alkyl;
R⁹ is hydrogen or C₁₋₄ alkyl;
R¹⁰ is hydrogen, C₁₋₄ alkyl, carbocyclyl or heterocyclyl; wherein
R¹⁰ is optionally substituted by one or more substituents selected from R¹⁹;
R¹¹ is carboxy, sulpho, sulphino, phosphono, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆ alkyl; or R¹¹ is a group of formula (I-2B″):

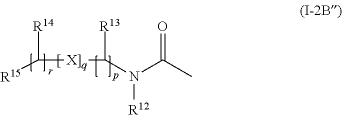

wherein
X is —N(R)—, —N(R^{q})C(O)—, —O—, or —S(O)ₐ— wherein a is 0 to 2 and R^{q} is hydrogen or C₁₋₄ alkyl;
R¹² is hydrogen or C₁₋₄ alkyl;
R¹³ and R¹⁴ are independently selected from hydrogen, C₁₋₄ alkyl, carbocyclyl and heterocyclyl; wherein R¹³ and R¹⁴ are optionally substituted by one or more substituents selected from R²⁰;
R¹⁵ is carboxy, sulpho, sulphino, phosphono, —P(O)(OR^{e})(OR^{f}), —P(O)(OH)(OR^{e}), —P(O)(OH)(R^{e}) and —P(O)(OR^{e})(R^{f}) wherein R^{e} and R^{f} are independently selected from C₁₋₆ alkyl;
p is 1-3; wherein the meanings of R¹³ may be the same or different;
q is 0-1;
r is 0-3; wherein the meanings of R¹⁴ may be the same or different;
m is 0-2; wherein the meanings of R¹⁰ may be the same or different;
n is 1-3; wherein the meanings of R⁷ may be the same or different;
R¹⁶, R¹⁷ and R¹⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl) amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R¹⁶, R¹⁷ and R¹⁸ may be independently optionally substituted on carbon by one or more R²¹;
R¹⁹ and R²⁰ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆ alkyl; wherein R¹⁹ and R²⁰ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl.

Hereinafter, in the case of indicating a compound of formula (I), it should be understood that the aspect thereof also relates to the compounds of formula (I-1) and the compounds of formula (I-2).

In addition, a person having ordinary skill in the art will recognize that the numbering system differs between the compounds of formula (I) and the compounds of formula (I-1). The numbering system used hereinafter in the present specification refers to the compounds of formula (I). However, it should be understood that such a numbering system is also applied to the meanings corresponding to those of formula (I-1).

In the present specification, the term “alkyl” includes both straight and branched chain alkyl groups, but the references to individual alkyl groups such as “propyl” are specific for the straight chain version only. For example, “C₁₋₆ alkyl” includes C₁₋₄ alkyl, C₁₋₃ alkyl, propyl, isopropyl and t-butyl. However, references to individual alkyl groups such as ‘propyl’ are specific for the straight-chain version only and references to individual branched chain alkyl groups such as ‘isopropyl’ are specific for the branched chain version only. A similar convention applies to other radicals, for example “phenyl C₁₋₆ alkyl” would include phenyl C₁₋₄ alkyl, benzyl, 1-phenylethyl and 2-phenylethyl. The term “halo” refers to fluoro, chloro, bromo and iodo.

In the case where optional substituents are chosen from “one or more” groups, it should be understood that such a definition includes all substituents being chosen from one of the specified groups or chosen from two or more of the specified groups.

“Heteroaryl” is a totally unsaturated, monocyclic or bicyclic ring containing 3-12 atoms of which at least one atom is chosen from nitrogen, sulfur or oxygen, and which may, unless otherwise specified, be carbon- and nitrogen-linked. A preferable “heteroaryl” refers to a totally unsaturated, monocyclic ring containing 5 or 6 atoms or a totally unsaturated, bicyclic ring containing 9 or 10 atoms of which at least one atom is chosen from nitrogen, sulfur and oxygen, and which may, unless otherwise specified, be carbon- or nitrogen-linked. In another aspect of the present invention, “heteroaryl” refers to a totally unsaturated, monocyclic ring containing 5 or 6 atoms or a totally unsaturated, bicyclic ring containing 8, 9 or 10 atoms of which at least one atom is chosen from nitrogen, sulfur and oxygen, and which may, unless otherwise specified, be carbon- or nitrogen-linked. Examples and suitable meanings of the term “heteroaryl” are thienyl, isoxazolyl, imidazolyl, pyrrolyl, thiadiazolyl, isothiazolyl, triazolyl, pyranyl, indolyl, pyrimidyl, pyrazinyl, pyridazinyl, pyridyl and quinolyl. The term “heteroaryl” preferably refers to thienyl or indolyl.

“Aryl” is a totally unsaturated, mono or bicyclic carbon ring that contains 3-12 atoms. Preferable “aryl” is a monocyclic ring containing 5 or 6 atoms or a bicyclic ring containing 9 or 10 atoms. Suitable meanings for “aryl” include phenyl or naphthyl. “Aryl” is more preferably phenyl.

“Heterocyclyl” is a saturated, partially saturated or unsaturated, mono or bicyclic ring containing 3-12 atoms of which at least one atom is chosen from nitrogen, sulfur and oxygen, and which may, unless otherwise specified, be carbon- or nitrogen-linked, wherein a —CH₂— group can optionally be replaced by a —C(O)—, or a ring sulfur atom may be optionally oxidized to form S-oxide. Preferably “heterocyclyl” is a saturated, partially saturated or unsaturated, mono or bicyclic ring containing 5 or 6 atoms of which at least one atom is chosen from nitrogen, sulfur and oxygen, and which may, unless otherwise specified, be carbon- or nitrogen-linked, wherein a —CH₂— group can optionally be replaced by a —C(O)— or a ring sulfur atom may be optionally oxidized to form S-oxide(s). Examples and suitable meanings of the term “heterocyclyl” are thiazolidinyl, pyrrolidinyl, pyrrolinyl, 2-pyrrolidonyl, 2,5-dioxopyrrolidinyl, 2-benzoxazolinonyl, 1,1-dioxotetrahydrothienyl, 2,4-dioxoimidazolidinyl, 2-oxo-1,3,4-(4-triazolinyl), 2-oxazolidinonyl, 5,6-dihydrouracilyl, 1,3-benzodioxolyl, 1,2,4-oxadiazolyl, 2-azabicyclo[2.2.1]heptyl, 4-thiazolidonyl, morpholino, 2-oxotetrahydrofuranyl, tetrahydrofuranyl, 2,3-dihydrobenzofuranyl, benzothienyl, tetrahydropyranyl, piperidyl, 1-oxo-1,3-dihydroisoindolyl, piperazinyl, thiomorpholino, 1,1-dioxothiomorpholino, tetrahydropyranyl, 1,3-dioxolanyl, homopiperazinyl, thienyl, isoxazolyl, imidazolyl, pyrrolyl, thiadiazolyl, isothiazolyl, 1,2,4-triazolyl, 1,3,4-triazolyl, pyranyl, indolyl, pyrinidyl, thiazolyl, pyrazinyl, pyridazinyl, pyridyl, 4-pyridonyl, quinolyl and 1-isoquinolonyl.

“Carbocyclyl” is a saturated, partially saturated or unsaturated, mono or bicyclic carbon ring that contains 3-12 atoms; wherein a —CH₂— group can optionally be replaced by a —C(O)—. Preferable “carbocyclyl” is a monocyclic ring containing 5 or 6 atoms or a bicyclic ring containing 9 or 10 atoms. Suitable meanings for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. In particular, “carbocyclyl” is cyclopropyl, cyclobutyl, 1-oxocyclopentyl, cyclopentyl, cyclopentenyl, cyclohexyl, cyclohexenyl, phenyl or 1-oxoindanyl.

An example of “C₁₋₆ alkanoyloxy” and “C₁₋₄ alkanoyloxy” is acetoxy. Examples of “C₁₋₆ alkoxycarbonyl” and “C₁₋₄ alkoxycarbonyl” include methoxycarbonyl, ethoxycarbonyl, and n- and t-butoxycarbonyl. Examples of “C₁₋₆ alkoxy” and “C₁₋₄ alkoxy” include methoxy, ethoxy and propoxy. Examples of “C₁₋₆ alkanoylamino” and “C₁₋₄ alkanoylamino” include formamido, acetamido and propionylamino. Examples of “C₁₋₆ alkyl S(O)ₐ wherein a is 0 to 2” and “C₁₋₄ alkyl S(O)ₐ wherein a is 0 to 2” include methylthio, ethylthio, methylsulphinyl, ethylsulphinyl, mesyl and ethylsulphonyl. Examples of “C₁₋₆ alkanoyl” and “C₁₋₄ alkanoyl” include C₁₋₃ alkanoyl, propionyl and acetyl. Examples of “N—(C₁₋₆ alkyl)amino” and “N—(C₁₋₄ alkyl)amino” include methylamino and ethylamino. Examples of “N, N—(C₁₋₆ alkyl)₂ amino” and “N, N—(C₁₋₄ alkyl)₂ amino” include di-N-methylamino, di-(N-ethyl)amino and N-ethyl-N-methylamino. Examples of “C₂₋₆ alkenyl” and “C₂₋₄ alkenyl” are vinyl, allyl and 1-propenyl. Examples of “C₂₋₆ alkynyl” and “C₂₋₄ alkynyl” are ethynyl, 1-propynyl and 2-propynyl. Examples of “N—(C₁₋₆ alkyl) sulphamoyl” and “N—(C₁₋₄ alkyl)sulphamoyl” are N—(C₁₋₃ alkyl)sulphamoyl, N-(methyl)sulphamoyl and N-(ethyl)sulphamoyl. Examples of “N—(C₁₋₆ alkyl)₂ sulphamoyl” and “N—(C₁₋₄ alkyl)₂ sulphamoyl” are N,N-(dimethyl)sulphamoyl and N-(methyl)-N-(ethyl)sulphamoyl. Examples of “N—(C₁₋₆ alkyl)carbamoyl” and “N—(C₁₋₄ alkyl)carbamoyl” are methylaminocarbonyl and ethylaminocarbonyl. Examples of “N,N—(C₁₋₆ alkyl)₂ carbamoyl” and “N,N—(C₁₋₄ alkyl)₂ carbamoyl” are dimethylaminocarbonyl and methylethylaminocarbonyl. Examples of “C₁₋₆ alkoxycarbonylamino” are ethoxycarbonylamino and t-butoxycarbonylamino. Examples of “N′—(C₁₋₆ alkyl)ureido” are N′-methylureido and N′-ethylureido. Examples of “N—(C₁₋₆ alkyl)ureido” are N-methylureido and N-ethylureido. Examples of “N′,N′—(C₁₋₆ alkyl)₂ ureido” are N′,N′-dimethylureido and N′-methyl-N′-ethylureido. Examples of “N′—(C₁₋₆ alkyl)-N—(C₁₋₆ alkyl)ureido” are N′-methyl-N-methylureido and N′-propyl-N-methylureido. Examples of “N′,N′—(C₁₋₆ alkyl)₂-N(C₁₋₆ alkyl)ureido” are N′,N′-dimethyl-N-methylureido and N′-methyl-N′-ethyl-N-propylureido.

A suitable pharmaceutically acceptable salt of the compound of the present invention mentioned above is, for example, an acid-addition salt of a compound of the present invention which is sufficiently basic, such as an acid-addition salt with, for example, an inorganic or organic acid, such as hydrochloric acid, hydrobromic acid, sulphuric acid, phosphoric acid, trifluoroacetic acid, citric acid or maleic acid. In addition, a suitable pharmaceutically acceptable salt of a compound of the present invention which is sufficiently acidic is an alkali metal salt such as a sodium or potassium salt, an alkaline earth metal salt such as a calcium or magnesium salt, an ammonium salt or a salt with an organic base which affords a physiologically-acceptable cation, for example, a salt with methylamine, dimethylamine, trimethylamine, piperidine, morpholine or tris-(2-hydroxyethyl)amine.

The compounds of formula (I) may be administered in the form of a pro-drug which is decomposed in the human or animal body to give a compound of formula (I). Examples of pro-drugs include in vivo hydrolysable esters and in vivo hydrolysable amides of a compound of formula (I).

An in vivo hydrolysable ester of a compound of formula (I) containing a carboxy or hydroxy group is, for example, a pharmaceutically acceptable ester which is hydrolyzed in the human or animal body to produce the parent acid or alcohol. Suitable pharmaceutically acceptable esters for carboxy include C₁₋₆ alkoxymethyl esters such as methoxymethyl, C₁₋₆ alkanoyloxymethyl esters such as pivaloyloxymethyl, phthalidyl esters, C₃₋₈ cycloalkoxycarbonyloxy C₁₋₆ alkyl esters such as 1-cyclohexylcarbonyloxyethyl; 1,3-dioxolen-2-onylmethyl esters such as 5-methyl-1,3-dioxolen-2-onylmethyl; and C₁₋₆ alkoxycarbonyloxyethyl esters such as 1-methoxycarbonyloxyethyl, and may be formed at any carboxy group in the compounds of the present invention.

An in vivo hydrolysable ester of a compound of formula (I) containing a hydroxy group includes inorganic esters such as phosphate esters and alpha-acyloxyalkyl ethers and related compounds which as a result of the in vivo hydrolysis of the ester decomposed to give the parent hydroxy group. Examples of alpha-acyloxyalkyl ethers include acetoxymethoxy and 2,2-dimethylpropionyloxy-methoxy. A selection of in vivo hydrolysable ester-forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. Examples of substituents on benzoyl include morpholino and piperazino linked from a ring nitrogen atom via a methylene group to the 3- or 4-position of the benzoyl ring.

A suitable meaning for an in vivo hydrolysable amide of a compound of formula (I) containing a carboxy group is, for example, an N—C₁₋₆ alkyl or N,N-di-C₁₋₆ alkyl amide such as N-methyl, N-ethyl, N-propyl, N,N-dimethyl, N-ethyl-N-methyl or N,N-diethyl amide.

The compounds mentioned above have IBAT inhibitory activities. Some compounds of formula (I) may have chiral centers and/or geometric isomeric centers (E- and Z-isomers), and it should be understood that the present invention encompasses all such optical, diastereoisomers and geometric isomers that possess IBAT inhibitory activities.

The aforementioned ingredient (A) in the present invention relates to any and all tautomeric forms of the compounds of formula (I) that possess IBAT inhibitory activities.

It should also be understood that certain compounds of formula (I) can exist in the solvated as well as unsolvated forms such as a hydrated form. It should be understood that the ingredient (A) mentioned above in the present invention encompasses all such solvated forms which possess IBAT inhibitory activities.

More preferable compounds as a compound of formula (I) are represented by the following formula (I-3):

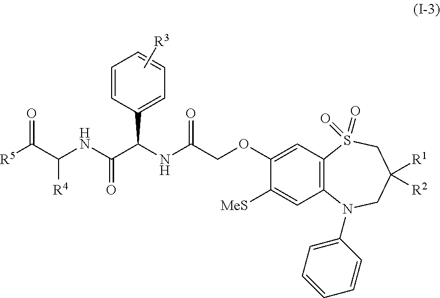

wherein:
R¹ and R² are independently selected from C₁₋₄ alkyl;
R³ is hydrogen, hydroxy or halo;
R⁴ is hydrogen or C₁₋₄ alkyl which may be substituted by hydroxy, methoxy and methyl S(O)ₐ wherein a is 0 to 2;
R⁵ is hydroxy or HOC(O)CH(R⁶)NH—;
R⁶ is selected from hydrogen and C₁₋₃ alkyl which may be substituted by hydroxy, methoxy and methyl S(O)ₐ wherein a is 0 to 2;
with the proviso that in the case where both R¹ and R² are butyl, R⁵ is hydroxy, and R⁴ is methylthiomethyl, methylsulfinylmethyl, 2-methylthioethyl, hydroxymethyl, or methoxymethyl, R³ is not hydrogen; and with the proviso that in the case where both R¹ and R² are butyl, R⁵ is HOC(O)CH(R⁶)NH—, and R⁶ is hydroxymethyl, and R⁴ is hydroxymethyl, R³ is not hydrogen.

As the compound of formula (I), in particular, 1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiadiazepine, that is, Elobixibat is preferable.

On the other hand, as the compound of formula (I′), in particular,
1,1-dioxo-3,3-dibutyl-5-phenyl-7-methylthio-8-(N—{(R)-α-[N-(2-sulphoethyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine; or
1,1-dioxo-3,3-dibutyl-5-phenyl-7-methylthio-8-(N—{(R)-α-[N—((S)-1-carboxypropyl)carbamoyl]-4-hydroxybenzyl}carbamoylmethoxy)-2,3,4,5-tetrahydro-1,2,5-benzothiadiazepine is preferable.

A compound of formula (I) or (I′), or a pharmaceutically acceptable salt, solvate, solvate of such a salt or a prodrug thereof can be manufactured in accordance with a method described in, for example, Japanese Patent No. 3665005 (the content of which is incorporated in the specification of the present application by reference).

The above-mentioned ingredient (A) possesses IBAT inhibitory activities. These properties may be assessed, for example, using an in vitro test assay for studying the effect on bile acid uptake in IBAT-transfected cells (Smith L., Price-Jones M. J., Hugnes K. T., and Jones N. R. A.; J Biomolecular Screening, 3, 227-230) or in vivo by studying the effect on radiolabelled bile acid absorption in mice/rats (Lewis M. C., Brieaddy L. E. and Root C., J., J Lip Res 1995, 36, 1098-1105).

The above-mentioned ingredient can be used in the treatment of dyslipidemic conditions and disorders such as hyperlipidemia, hypertrigliceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL) in a warm-blooded animal, such as a human being.

In addition, the above-mentioned ingredient (A) can be used in the treatment of different clinical conditions such as atherosclerosis, arteriosclerosis, arrhythmia, hyper-thrombotic conditions, vascular dysfunction, endothelial dysfunction, heart failure, coronary heart diseases, cardiovascular diseases, myocardial infarction, angina pectoris, peripheral vascular diseases, inflammation of cardiovascular tissues such as heart, valves, vasculature, arteries and veins, aneurisms, stenosis, restenosis, vascular plaques, vascular fatty streaks, leukocyte, monocytes and/or macrophage infiltrate, intimital thickening, medial thinning, infectious and surgical trauma and vascular thrombosis, stroke and transient ischemic attacks in a warm-blooded animal, such as a human being.

There is evidence indicating that an IBAT inhibitor can be potentially useful in the treatment and/or prevention of gallstone or cholelithiasis. The above-mentioned ingredient (A) can be used in the treatment and/or prevention of gallstone or cholelithiasis in a warm-blooded animal, such as a human being.

In the present invention, the above-mentioned ingredient (A) can also be used in the treatment of gastrointestinal disorders. In particular, the ingredient (A) can be used in the treatment of chronic constipation, functional constipation and irritable bowel syndrome, and in particular, constipation-dominant irritable bowel syndrome (C-IBS).

In the specification of the present application, in the case of using the terms “functional constipation” and “C-IBS”, it should be understood that they are defined in accordance with “Rome 2 Criteria” (Gut 45 (Suppl 2): 43, 1999, II43-II47).

An amount of the above-mentioned ingredient (A) contained in the solid formulation of the present invention is not particularly limited, and can range from 0.01 to 50% by weight, preferably ranges from 0.05 to 40% by weight, more preferably ranges from 0.1 to 30% by weight, even more preferably ranges from 0.2 to 20% by weight, even more preferably ranges from 0.5 to 10% by weight, and in particular, preferably ranges from 0.8 to 5% by weight, based on the total weight of the solid formulation.

An amount of the above-mentioned ingredient (A) contained in the solid formulation of the present invention is not particularly limited, and can range from 0.1 to 100 mg, preferably ranges from 0.3 to 75 mg, more preferably ranges from 0.5 to 50 mg, even more preferably ranges from 0.8 to 30 mg, and in particular, preferably ranges from 1 to 20 mg.

The aforementioned plasticizer usable in the present invention is (B) at least one selected from the group consisting of polyethylene glycol, propylene glycol, glycerol, glyceryl triacetate, triethyl acetylcitrate, dibutyl sebacate, diethyl phthalate, castor oil, a copolymer of propylene oxide and ethylene oxide, triacetin, triethyl citrate, and a mixture thereof (hereinafter, simply referred to as “ingredient (B)” in some cases).

An average molecular weight of the polyethylene glycol as ingredient (B) preferably ranges from 200 to 20,000, more preferably ranges from 300 to 10,000, and even more preferably ranges from 400 to 6,000. The average molecular weight used herein may be a number average molecular weight.

A particular mode of isolation in the case of isolating ingredient (A) from ingredient (B) is not particularly limited, and any options can be used as long as direct contact between ingredient (A) and ingredient (B) is inhibited. For example, at least one isolation layer can be provided between ingredient (A) and ingredient (B).

A material of the isolation layer mentioned above is not particularly limited, as long as the material does not include ingredient (B). For example, the material of the isolation layer may be a water-soluble polymer of a cellulose derivative such as hydroxypropylmethylcellulose (hypromellose) or hydroxypropylcellulose, a water-soluble vinyl derivative (such as polyvinyl alcohol), or a water-soluble polymer such as starch. In addition, as a material of the isolation layer mentioned above, a lubricant such as calcium stearate, glycerol monostearate, glyceryl palmitostearate, magnesium stearate, sodium stearyl fumarate, sucrose fatty acid ester, zinc stearate, stearic acid, or talc can also be used. In addition, a coating agent such as titanium oxide can also be used. In order to achieve assured isolation, a water-soluble polymer is preferably used, and use of hypromellose is more preferable.

Weight of the isolation layer mentioned above is not particularly limited. An amount of the isolation layer preferably ranges from 0.1 to 20% by weight, more preferably ranges from 0.5 to 15% by weight, and even more preferably ranges from 1 to 10% by weight, based on the total weight of the solid formulation.

Thickness of the isolation layer mentioned above is not particularly limited, and preferably ranges from 0.01 to 5 mm, more preferably ranges from 0.05 to 3 mm, and even more preferably ranges from 0.1 to 1 mm.

In the case of isolating ingredient (A) from ingredient (B), an amount of ingredient (B) contained in a solid formulation of the present invention is not limited.

On the other hand, in the case of ingredient (A) not being isolated from ingredient (B), that is, in the case of ingredient (A) contacting ingredient (B), in a certain embodiment, an amount of ingredient (B) in the solid formulation of the present invention is 0.9% by weight or less based on the total weight of the solid formulation. An amount of ingredient (B) in the solid formulation of the present invention is preferably 0.8% by weight or less, more preferably 0.6% by weight or less, even more preferably 0.4% by weight or less, and in particular, preferably 0.3% by weight, based on the total weight of the solid formulation.

In the case of ingredient (A) not being isolated from ingredient (B), that is, in the case of ingredient (A) contacting ingredient (B), in another embodiment, the solid formulation of the present invention contains at least one core, and at least one coating layer or capsule layer enclosing at least a part of the core, where the core contains ingredient (A), the coating layer or capsule layer contains ingredient (B), in which an amount of ingredient (B) in the coating layer or capsule layer is 45% by weight or less based on the total weight of the coating layer or capsule layer. The amount of ingredient (B) in the coating layer or capsule layer is preferably 40% by weight or less, more preferably ranges from 0.1 to less than 40% by weight, even more preferably ranges from 1 to 35% by weight, and even more preferably ranges from 5 to 10% by weight, based on the total weight of the coating layer or capsule layer. The aforementioned “layer” is the coating layer or the capsule layer, and is not the isolation layer.

In the case of ingredient (A) being isolated from ingredient (B), an amount of ingredient (B) contained in the solid formulation of the present invention can range from 0.1 to 40% by weight based on the total weight of ingredient (A). On the other hand, in the case of ingredient (A) being not isolated from ingredient (B), an amount of ingredient (B) contained in the solid formulation of the present invention may range, for example, from 0.1 to 20% by weight, and may range from 1 to 15% by weight or from 5 to 10% by weight based on the total weight of ingredient (A).

Only one core may be present in the solid formulation of the present invention, or two or more cores may be present. In the case of the solid formulation of the present invention containing the aforementioned core, the core contains ingredient (A). On the other hand, the aforementioned core preferably does not contain ingredient (B). The form of the core is not particularly limited, and the core may be in the form of a mixture of simple powders, granules or the like. On the other hand, in the case of the solid formulation of the present invention being in the form of a film coating tablet, the core mentioned above can be an uncoated tablet before film-coating. In addition, in the case of the solid formulation of the present invention being in the form of a capsule tablet, the aforementioned core can form a granule to be capsulated.

The aforementioned core preferably contains an inert carrier together with ingredient (A). The inert carrier mentioned above preferably contains at least one additive selected from the group consisting of a filler, a disintegrant, a binder, a lubricant, and a fluidizer.

As the filler, at least one selected from the group consisting of sugars, sugar alcohols, inorganic fillers and crystalline cellulose is preferable. Examples of sugars include, for example, lactose (lactose hydrate, anhydrous lactose), saccharose, sucrose, fructose, fructooligosaccharides, glucose, maltose, reduced maltose, powder sugar, powdered candy, reduced lactose, and the like. Examples of sugar alcohols include, for example, erythritol, sorbitol, maltitol, xylitol, mannitol, and the like. Examples of inorganic fillers include, for example, anhydrous calcium hydrogen phosphate, anhydrous calcium phosphate, precipitated calcium carbonate, calcium silicate, and the like. A combination of two or more types among these may be used. Mannitol, crystalline cellulose, or a mixture thereof is preferable. An amount of the filler in the core mentioned above is not particularly limited, usually ranges from 60 to 99% by weight, preferably ranges from 70 to 95% by weight, and more preferably ranges from 80 to 90% by weight based on the total weight of the core.

As the disintegrant, at least one selected from the group consisting of natural starches, starch derivatives, crospovidone, carboxymethyl cellulose, carboxymethyl cellulose calcium, low-substituted hydroxypropyl cellulose and carmellose is preferable. For example, examples of natural starches include corn starch, potato starch, rice starch, wheat starch and the like. Examples of starch derivatives include hydroxypropyl starch obtained by processing the natural starch, and the like. A combination of two or more types among these may be used. Carmellose is preferable, croscarmellose is more preferable, and croscarmellose sodium is even more preferable. An amount of the disintegrant in the aforementioned core is not particularly limited, but usually ranges from 0.1 to 20% by weight, preferably ranges from 1.0 to 10% by weight, and more preferably ranges from 2.0 to 5% by weight based on the total weight of the core.

Examples of the binder include, for example, hydroxypropylcellulose, polyvinyl alcohol, povidone (polyvinylpyrrolidone), hypromellose (hydroxypropylmethylcellulose), agar, gelatin and the like. A combination of two or more types among these may be used. Hypromellose is preferable. An amount of the binder in the aforementioned core is not particularly limited, but usually ranges from 0.1 to 20% by weight, preferably ranges from 1.0 to 10% by weight, and more preferably ranges from 2.0 to 5% by weight.

Examples of the lubricant include, for example, calcium stearate, glycerol monostearate, glyceryl palmitostearate, magnesium stearate, sodium stearyl fumarate, sucrose fatty acid ester, zinc stearate, stearic acid, talc, and the like. A combination of two or more types among these may be used. Magnesium stearate is preferable. An amount of the lubricant in the core is not particularly limited, but usually ranges from 0.1 to 20% by weight, preferably ranges from 0.5 to 10% by weight, and more preferably ranges from 1.0 to 5% by weight based on the total weight of the core.

Examples of fluidizer include, for example, light anhydrous silicic acid, hydrated silicon dioxide, and the like. A combination of two or more types among these may be used. Light anhydrous silicic acid is preferable. An amount of the fluidizer in the core is not particularly limited, but usually ranges from 0.01 to 10% by weight, preferably ranges from 0.1 to 5% by weight, and more preferably ranges from 0.5 to 3% by weight based on the total weight of the core.

In order to provide good administration ability in the oral cavity, a sweetener and/or a flavoring agent or a perfuming agent may preferably be added to the aforementioned core. Examples of the sweetener include, for example, dipotassium glycyrrhizinate, saccharin sodium, saccharin, stevia, aspartame, sucralose, thaumatin, acesulfame-K, neotame, and the like. Examples of the flavoring agent or the perfuming agent include, for example, citrus flavors of lemon, orange, grapefruit, and the like, peppermint, spearmint, menthol, pine, cherry, fruit, yogurt, coffee, and the like.

In the core mentioned above, non-toxic and inert additives commonly used in the formulation field can be added within the range which does not affect the effects of the present invention. Examples of the additive used include, for example, a surfactant, an organic acid, a colorant, and the like.

A method for producing the core mentioned above is not particularly limited. For example, in the case of the core mentioned above being in the form of a granule, the core can be produced by means of a fluid bed granulator represented by a flow coater (manufactured by Freund Corp.), a GPCG (Glatt Powder Coater Granulator), a WSG (Wirbel Schicht Granulator), a multiplex (GLATT/manufactured by Powrex Corporation), or the like, or by means of a stirring granulator represented by a vertical granulator (manufactured by Powrex Corporation), or the like.

In addition, in the case of the core mentioned above being in the form of an uncoated tablet, a wet granulation tableting method in which the granules manufactured by means of the aforementioned manufacturing method are molded, a direct tableting method in which various raw materials are suitably mixed, and the mixed powder is molded, or a dry granulation tableting method can be used. As the molding method mentioned above, a compression molding method using a rotary tableting machine or the like is preferable from a commercial point of view. The uncoated tablet can also be molded by means of an external lubricating method. In this case, tableting is carried out after mixing the ingredients other than a lubricant, while spraying the lubricant on a die-punch, or alternatively, tableting is carried out after previously mixing a part of the lubricant with the ingredients other than a lubricant, by spraying the remaining lubricant on a die-punch. In addition, the uncoated tablet can also be produced by means of a special tablet press such as a tablet press for nucleated tablets, a two-layer tablet press, or a three-layer tablet press.

In the case of the core mentioned above being an uncoated tablet, in order to maintain a good balance between disintegration time and hardness, a suitable tableting pressure is preferably selected during production of the uncoated tablets. The tableting pressure is normally 2 kN (about 200 kgf) or more, preferably 4 kN (about 400 kgf) or more, and more preferably 6 kN (about 600 kgf) or more.

In the solid formulation of the present invention according to the aforementioned embodiment, only one coating layer or capsule layer mentioned above enclosing the core mentioned above may be present, or two or more coating layers or capsule layers may be present. Here, “enclosing” means that the coating layer or capsule layer encloses the core, and does not necessarily contact the core. For example, at least one isolating layer may be present between the core and the coating layer or capsule layer. In this case, the core does not directly contact the coating layer or capsule layer.

The weight of the isolation layer mentioned above is not particularly limited, and preferably ranges from 0.1 to 20% by weight, more preferably ranges from 0.5 to 15% by weight, and even more preferably ranges from 1 to 10% by weight based on the total weight of the solid formulation.

The coating layer or capsule layer mentioned above can be present in a ratio ranging from 0.1 to 20% by weight, preferably ranging from 0.5 to 15% by weight and further more ranging from 1 to 10% by weight based on the total weight of the solid formulation. The coating layer or capsule layer mentioned above may include a small amount of ingredient (A). In this case, an amount of the aforementioned ingredient (A) included therein is preferably 10% by weight or less, more preferably 5% by weight or less, more preferably 1% by weight or less, and even more preferably 0.1% by weight or less, based on the total weight of the aforementioned layer. In particular, preferably, the coating layer or capsule layer mentioned above does not contain ingredient (A).

Preferably, the solid formulation of the present invention contains:
at least one core, and
at least one coating layer or capsule layer enclosing at least a part of the core,
where:
the core contains the ingredient (A) mentioned above,
the coating layer or capsule layer contains the ingredient (B) mentioned above, and
at least one isolation layer is provided between the core and the coating layer or capsule layer, or
in the case of the core contacting the coating layer or capsule layer,
an amount of the ingredient (B) mentioned above in the coating layer or capsule layer is 0.9% by weight or less based on the total weight of the solid formulation, or
an amount of the ingredient (B) mentioned above in the coating layer or capsule layer is 45% by weight or less based on the total weight of the coating layer or capsule layer.

In this case, an amount of the ingredient (B) mentioned above in the coating layer or capsule layer mentioned above is preferably 0.8% by weight or less, more preferably 0.6% by weight or less, even more preferably 0.4% by weight of less, and in particular, preferably 0.3% by weight or less based on the total weight of the solid formulation.

In addition, in this case, an amount of the ingredient (B) mentioned above in the coating layer or capsule layer mentioned above is preferably 40% by weight or less, more preferably ranges from 0.1 to less than 40% by weight, even more preferably ranges from 1 to 35% by weight, and even more preferably ranges from 5 to 10% by weight based on the total weight of the coating layer or capsule layer mentioned above.

In the present invention, if the amount of the ingredient (B) mentioned above in the coating layer or capsule layer is 0.9% by weight or less based on the total weight of the solid formulation, the amount of the ingredient (B) mentioned above in the coating layer or capsule layer may exceed 45% by weight based on the total weight of the coating layer or capsule layer.

On the other hand, if the amount of the ingredient (B) mentioned above in the coating layer or capsule layer is 45% by weight or less based on the total weight of the coating layer or capsule layer, the amount of the ingredient (B) mentioned above in the coating layer or capsule layer may exceed 0.9% by weight based on the total weight of the solid formulation. In this case, the amount of the ingredient (B) mentioned above in the coating layer or capsule layer may be, for example, 3.0% by weight or less, is preferably 2.0% by weight or less, is more preferably 1.5% by weight or less, and is even more preferably 1.4% by weight or less based on the total weight of the solid formulation. In particular, the amount of the ingredient (B) mentioned above in the coating layer or capsule layer is preferably 1.4% by weight or less based on the total weight of the solid formulation, and more preferably ranges from 0.1 to less than 40% by weight, even more preferably ranges from 1 to 35% by weight, and even more preferably ranges from 5 to 10% by weight based on the total weight of the coating layer or capsule layer.

A more preferable embodiment of the solid formulation of the present invention contains:
at least one core, and
at least one coating layer or capsule layer enclosing at least a part of the core,
where:
the core contains the ingredient (A) mentioned above,
the coating layer or capsule layer contains the ingredient (B) mentioned above, and
at least one isolation layer is provided between the core and the coating layer or capsule layer, or
in the case of the core contacting the coating layer or capsule layer,
an amount of the ingredient (B) mentioned above in the coating layer or capsule layer is 0.9% by weight or less, preferably 0.8% by weight or less, more preferably 0.6% by weight or less, even more preferably 0.4% by weight or less, and in particular, preferably 0.3% by weight or less based on the total weight of the solid formulation, and
an amount of the ingredient (B) mentioned above in the coating layer or capsule layer is 45% by weight or less, is preferably 40% by weight or less, more preferably ranges from 0.1 to less than 40% by weight, even more preferably ranges from 1 to 35% by weight, and in particular, preferably ranges from 5 to 10% by weight based on the total weight of the coating layer or capsule layer.

In this case, the amount of the ingredient (B) mentioned above in the coating layer or capsule layer may correspond to any one of combinations 1 to 25 shown in the following table. The combinations 6 to 25 are preferable, the combinations 11 to 25 are more preferable, the combinations 16 to 25 are even more preferable, the combinations 21 to 25 are even more preferable, and the combination 25 is particularly preferable.

| | | Based on the total weight of the solid formulation | | | | |
|---|---|---|---|---|---|---|
| | | 0.9% by | 0.8% by | 0.6% by | 0.4% by | 0.3% by |
| | | weight | weight | weight | weight | weight |
| | | or less | or less | or less | or less | or less |
| Based on | 45% by | 1 | 2 | 3 | 4 | 5 |
| the total | weight | | | | | |
| weight of | or less | | | | | |
| the layer | 40% by | 6 | 7 | 8 | 9 | 10 |
| | weight or | | | | | |
| | less | | | | | |
| | 0.1 to less | 11 | 12 | 13 | 14 | 15 |
| | than 40% | | | | | |
| | by weight | | | | | |
| | 1 to 35% | 16 | 17 | 18 | 19 | 20 |
| | by weight | | | | | |
| | 5 to 10% | 21 | 22 | 23 | 24 | 25 |
| | by weight | | | | | |

The coating layer or capsule layer preferably further contains at least one selected from the group consisting of a water-soluble polymer other than polyethylene glycol, a colorant, a lubricant, and wax.

Examples of the water-soluble polymer mentioned above include, for example, cellulose-based derivatives such as hypromellose (hydroxypropyl methylcellulose), methyl cellulose, hydroxypropyl cellulose, carboxymethyl cellulose sodium, hydroxyethyl methylcellulose, hydroxypropyl methylcellulose phthalate, and hydroxypropyl methylcellulose acetate succinate, starches such as starch and pullulan, water-soluble vinyl derivatives such as polyvinyl alcohol and polyvinyl pyrrolidone, sodium alginate, gum arabic powder, gelatin and the like. Hypromellose, hydroxypropyl cellulose, water-soluble vinyl derivatives, and starches are preferable. Hypromellose, hydroxypropyl cellulose, and water-soluble vinyl derivatives are more preferable. Hypromellose and hydroxypropyl cellulose are most preferable. In addition, a mixture of a disintegrant auxiliary agent and an enteric polymer or a water-insoluble polymer may be contained, in addition to the water-soluble polymer. Examples of the enteric polymer include, for example, enteric cellulose esters such as cellulose acetate propionate, hydroxypropyl methylcellulose acetate succinate (for example, trade name: Shin-Etsu AQOAT, manufactured by Shin-Etsu Chemical Co., Ltd.), hydroxypropyl methylcellulose phthalate, carboxymethyl ethylcellulose, and cellulose acetate phthalate, enteric acrylic acid-based copolymers such as methacrylic acid copolymer L (for example, trade name: Eudragit L, manufactured by Evonik Degussa Japan), methacrylic acid copolymer LD (for example, trade name: Eudragit L 30D-55, manufactured by Evonik Degussa Japan, tradename: POLYQUID PA 30, POLYQUID PA 30-S, manufactured by Sanyo Chemical Industries, Ltd., trade name: Kollicoat MAE 30DP, manufactured by BASF), and methacrylic acid copolymer S (for example, trade name: Eudragit S, Eudragit S 100, Eudragit FS 30D, manufactured by Evonik Degussa Japan), and the like. These polymers may be used in a mixture of two or more types.

Polyvinyl alcohol is not particularly limited as long as it can be usually used in a film coating of a pharmaceutical product, and may be a fully saponified product or a partially saponified product. As the partially saponified product, for example, a product having a degree of saponification ranging from 70 to 95% by mol, in particular, ranging from 80 to 90% by mol, and even particularly 85 to 90% by mol is preferably used. In addition, a degree of polymerization is not particularly limited, preferably ranges from 100 to 3,000 and more preferably ranges from 300 to 1,000.

The water-soluble polymer is preferably hydroxypropylmethylcellulose. An amount of the water-soluble polymer mentioned above in the coating layer or capsule layer mentioned above is not particularly limited. The amount usually ranges from 50 to 99% by weight, preferably ranges from 60 to 95% by weight and more preferably ranges from 70 to 90% by weight based on the total weight of the coating layer or capsule layer.

The colorant mentioned above is preferably selected from the group consisting of titanium oxide, iron oxide, zinc oxide, tar pigments, and lake pigments.

Examples of iron oxide include, for example, black iron oxide, red ferric oxide, yellow ferric oxide, and the like. Examples of tar pigments include, for example, water-soluble edible tar pigments such as food yellow No. 5 and food blue No. 2. Examples of lake pigments include, for example, yellow No. 5 aluminum lake, and the like. A combination of two or more types among these may be used. Titanium oxide is preferable. An amount of the colorant in the coating layer or capsule layer mentioned above is not particularly limited, but usually ranges from 1 to 20% by weight, preferably ranges from 3 to 15% by weight, and more preferably ranges from 5 to 10% by weight based on the total weight of the coating layer or capsule layer.

Examples of the lubricant include, for example, calcium stearate, glycerol monostearate, glyceryl palmitostearate, magnesium stearate, sodium stearyl fumarate, sucrose fatty acid ester, zinc stearate, stearic acid, talc, and the like. A combination of two or more types among these may be used. Talc is preferable.

An amount of the lubricant in the coating layer and capsule layer is not particularly limited, but usually ranges from 0.1 to 20% by weight, preferably ranges from 0.5 to 15% by weight, and more preferably ranges from 1.0 to 10% by weight based on the total weight of the coating layer or capsule layer.

Examples of the wax include, for example, carnauba wax, beeswax, stearic acid and the like. A combination of two or more types among these may be used. Carnauba wax is preferable. An amount of the wax in the coating layer or capsule layer is not particularly limited, but usually ranges from 0.01 to 10% by weight, preferably ranges from 0.05 to 1% by weight, and more preferably ranges from 0.05 to 0.1% by weight based on the total weight of the coating layer or capsule layer.

The coating layer or capsule layer can contain a plasticizer other than the ingredient (B) mentioned above. Examples of the plasticizer mentioned above include, for example, polysorbates such as polysorbate 20, polysorbate 40, polysorbate 60, and polysorbate 80, and the like. An amount of the aforementioned plasticizer ranges, for example, from 1 to 20% by weight, preferably ranges from 3 to 15% by weight, and more preferably ranges from 5 to 10% by weight based on the coating layer or capsule layer mentioned above.

In order to further prevent or reduce the coloration over time of the solid formulation of the present invention, in the case of the core mentioned above contacting the coating layer or capsule layer mentioned above, the coating layer or capsule layer preferably does not contain a combination of polyethylene glycol and polyvinyl alcohol, or alternatively, in the case of the coating layer or capsule layer mentioned above containing a combination of polyethylene glycol and polyvinyl alcohol, the core is preferably isolated from the coating layer or capsule layer by means of at least one isolation layer.

A method for forming the coating layer or capsule layer is not particularly limited. In the case of the core directly contacting the coating layer or capsule layer, the coating layer or capsule layer may be directly formed on the surface of the core by means of a coating machine represented by HICOATER, new HICOATER, AQUA COATER (manufactured by Freund Corp.), DOREA COATER, POWREX COATER (manufactured by Powrex Corporation), or the like, a sugar-coating pan, a Wurster type coating machine, or the like. On the other hand, in the case of the core directly non-contacting the coating layer or capsule layer, at least one isolation layer mentioned above may be formed on the surface of the core by means of the aforementioned coating machine, and the coating layer or capsule layer can be formed on the surface of the isolation layer by means of the aforementioned coating machine. In addition, after forming the coating layer or capsule layer, an intraoral disintegration property can also be improved by humidification or the like.

The formation of the coating layer or capsule layer and the formation of the isolation layer are preferably carried out using an aqueous coating liquid. The aqueous coating liquid means an aqueous dispersion or solution of the constitutional ingredients of the coating layer or capsule layer or those of the isolation layer, and means a coating liquid containing of water or a mixed solution of water/water-soluble organic solvent, as a medium.

The water amount in the aqueous coating liquid is suitably determined in accordance with types and blending amounts of the ingredients and the amount of the water-soluble organic solvent added. The preferable water amount ranges, for example, from 5 to 1,000 parts by weight, preferably ranges from 7 to 100 parts by weight, and more preferably ranges from 8 to 50 parts by weight based on one part by weight of the constitutional ingredients of the isolation layer or the coating layer or capsule layer.

Examples of the water-soluble organic solvent which may be added to the aqueous coating liquid include, for example, methanol, ethanol, propyl alcohol, isopropyl alcohol, acetone, methyl ethyl ketone, dioxane, tetrahydrofuran, acetonitrile, and the like. In particular, ethanol is preferable. The amount of the water-soluble organic solvent added is determined in accordance with the types and blending amount of the ingredients, preferably ranges from 0 to 8.0 parts by weight, more preferably ranges from 0 to 2.4 parts by weight, even more preferably ranges from 0 to 1.3 parts by weight, and even more preferably ranges from 0 to 0.4 parts by weight based on one part by weight of water. In particular, the medium is preferably only water without adding a water-soluble organic solvent. Here, the medium which is only water means that only water is substantially used, and contamination of a small amount (for example 0.03 parts by weight or less based on one part by weight of water) of an organic solvent is acceptable.

The temperature of the exhaust gas temperature of the coating machine during the coating step is preferably controlled so that the temperature is higher than 30° C., but lower than 60° C. in the present invention. The coating step used herein means a step of applying the coating liquid on the core by means of a spray or the like, and during the step, ventilation is carried out. The exhaust gas temperature mentioned above is preferably 32° C. or higher, but 55° C. or lower, and more preferably 35° C. or higher, but 45° C. or lower. When the exhaust gas temperature mentioned above is 30° C. or lower, or alternatively 60° C. or higher, separation of a coating film may easily occur, roughness of the coating film may be increased, and therefore, a good coating film may not be formed in some cases.

Alternatively, in the present invention, the product temperature during the coating step is preferably controlled so that the product temperature is higher than 20° C., but lower than 56° C. Here, the product temperature during the coating step is the temperature of the core during the coating step. The product temperature can be measured by means of an infrared thermometer. The product temperature mentioned above is preferably 25° C. or higher, but 50° C. or lower, and more preferably 35° C. or higher, but 45° C. or lower. If the aforementioned product temperature is 20° C. or lower or 56° C. or higher, separation of the coating film may easily occur, roughness of the coating film may be increased, and a good coating film may not be formed in some cases.

During controlling of the exhaust gas temperature or the product temperature, the adjustment of the exhaust gas temperature or the product temperature can be carried out by adjusting, for example, the charge gas temperature, the amount of the charge gas, or the addition rate of the coating liquid (spraying rate and the like). In particular, controlling of the charge gas temperature is preferably carried out.

The application of the coating liquid may be carried out by pouring-and-adding or spraying, and spraying is preferable. In the case where, for example, 1 kg of uncoated tablets (250 mg/tablet) are subjected to spray coating by means of a ventilation type coating machine such as HICOATER (manufactured by Freund Corp.) or the like, the blast temperature may be set based on the exhaust temperature criteria, and the spray coating can be carried out in an air volume ranging from 1.5 to 3.5 m³/min at a spray rate ranging from 5 to 50 g/min.

The particular structure of the solid formulation of the present invention is not particularly limited. For example, the solid formulation may be in the form of fine granules, granules, capsules, or tablets. In the case of tablets, from one to two dividing lines for making division of the tablet easy may be provided. The shape of the tablet is not particularly limited, and may be, for example, round, oval (any oblong except for perfect circle; oval, egg-shaped, elliptical cylinder shape, old gold coin-shaped, or the like), diamond-shaped, triangle, or the like. The solid formulation may be in the form of so-called specially shaped tablets. In the case of providing a dividing line, the dividing line shape may be any of flat groove type, U-shaped groove type, or V-groove type. In the case of the tablet being in an oval shape, the dividing line can be preferably formed along the minor axis of the tablet.

The solid formulation of the present invention is preferably in the form of a tablet or a capsule. In the case of a tablet, a film-coating agent is preferable.

The size of the tablet mentioned above is not particularly limited. In the case of the tablet being in a general-column shape, the diameter of the column preferably ranges from 5 to 11 mm, more preferably ranges from 5 to 10 mm, and even more preferably ranges from 5 to 9 mm. In the case of the tablet being a specially shaped tablet, the maximum length of the specially shaped tablet can range from 5 to 11 mm, more preferably ranges from 5 to 10 mm, and even more preferably ranges from 5 to 9 mm.

A second aspect of the present invention relets to a method for stabilizing a certain benzothia (dia) zepine derivative in a solid formulation containing the same and a specific plasticizer, wherein the method includes:
isolating the benzothia(dia)zepine derivative mentioned above from the plasticizer mentioned above; or
in the case of the benzothia(dia)zepine derivative mentioned above not being isolated from the plasticizer mentioned above,
controlling an amount of the plasticizer to 0.9% by weight or less based on the total weight of the solid formulation, or
providing the solid formulation with a form containing at least one core and at least one coating layer or capsule layer enclosing at least a part of the core,
mixing the above-mentioned ingredient (A) into said core,
mixing the above-mentioned ingredient (B) into said coating layer or capsule layer, and
controlling an amount of the above-mentioned ingredient (B) in the coating layer or capsule layer to 45% by weight or less based on the total weight of the coating layer or capsule layer.

The aforementioned benzothia(dia)zepine derivative is identical to the aforementioned ingredient (A) in the first aspect of the present invention. Therefore, hereinafter, it is referred to as ingredient (A).

The aforementioned specific plasticizer is identical to the aforementioned ingredient (B) in the first aspect of the present invention. Therefore, hereinafter, it is referred to as ingredient (B).

In the case of the aforementioned ingredient (A) being isolated from the aforementioned ingredient (B), the amount of the aforementioned ingredient (B) contained in the solid formulation of the present invention is not limited.

On the other hand, in the case of the aforementioned ingredient (A) not being isolated from the aforementioned ingredient (B), that is, in the case of the aforementioned ingredient (A) contacting the aforementioned ingredient (B), in a certain embodiment, the amount of the aforementioned ingredient (B) in the solid formulation of the present invention is controlled to 0.9% by weight or less, preferably 0.8% by weight, more preferably 0.6% by weight, even more preferably 0.4% by weight, and even more preferably 0.3% by weight, based on the total weight of the solid formulation.

In the case where the core contains ingredient (A) and the coating layer or capsule layer contains the aforementioned ingredient (B), the amount of the ingredient (B) mentioned above in the coating layer or capsule layer may be controlled to 0.9% by weight or less based on the total weight of the solid formulation. In this case, the amount of the ingredient (B) mentioned above in the coating layer or capsule layer is preferably 0.8% by weight or less, preferably 0.6% by weight or less, even more preferably 0.4% by weight or less, and in particular, preferably 0.3% by weight of less, based on the total weight of the solid formulation.

In addition, in the case of the core containing the aforementioned ingredient (A), and the coating layer or capsule layer containing the aforementioned ingredient (B), the amount of the ingredient (B) in the coating layer or capsule layer may be 45% by weight or less based on the total weight of the coating layer or capsule layer. In this case, the amount of the ingredient (B) in the coating layer or capsule layer is preferably 40% by weight or less, more preferably ranges from 0.1 to less than 40% by weight, even more preferably ranges from 1 to 35% by weight, and even more preferably ranges from 5 to 10% by weight based on the total weight of the coating layer or capsule layer.

The explanation described above for the first aspect of the present invention can be applied to the solid formulation in the second aspect of the present invention. Therefore, for example, the amount of ingredient (B) in the solid formulation may range from 0.1 to 20% by weight and may range from 1 to 15% by weight or from 5 to 10% by weight based on the total weight of the ingredient (A).

In addition, the amount of the ingredient (A) mentioned above in the solid formulation is not particularly limited, can range from 0.1 to 100 mg, preferably ranges from 0.3 to 75 mg, more preferably ranges from 0.5 to 50 mg, even more preferably ranges from 0.8 to 30 mg, and in particular, preferably ranges from 1 to 20 mg.

In addition, the coating layer or capsule layer can exist in a ratio ranging from 0.1 to 20% by weight, preferably ranges from 0.5 to 15% by weight, and more preferably ranges from 1 to 10% by weight based on the total weight of the solid formulation.

In accordance with the present invention, in a solid formulation containing ingredient (A), the ingredient (A) mentioned above can be stabilized therein. Therefore, a solid formulation containing stabilized ingredient (A) can be provided.

Ingredient (A) in the solid formulation mentioned above is stable over time even under an atmosphere of high temperature and/or high humidity. Therefore, even if the solid formulation of the present invention is present under an atmosphere of high temperature and/or high humidity, generation of related substance(s) derived from decomposition of the ingredient (A) mentioned above can be prevented or reduced. In particular, in accordance with the present invention, the solid formulation can be stable under an open atmosphere. An amount of the related substance(s) in the solid formulation of the present invention is preferably 3.0% by weight or less, more preferably 2.5% by weight or less, and even more preferably 2.0% by weight or less based on the total weight of the solid formulation.

Therefore, in accordance with the present invention, the solid formulation can be stored for a long period of time, and the pharmaceutical effects of the ingredient (A) mentioned above contained in the solid formulation can be maintained. In particular, in accordance with the present invention, the solid formulation containing the ingredient (A) mentioned above can be stable even under an atmosphere of high temperature and high humidity in the summer season.

### INDUSTRIAL APPLICABILITY

The present invention can provide a stabilized solid formulation containing a specified benzothia(dia)zepine derivative. The specific benzothia(dia)zepine derivatives mentioned above can function as an IBAT inhibitor, and for this reason, the solid formulations according to the present invention are useful for a long period of time in the treatment of dyslipidemic conditions and disorders such as hyperlipidemia, hypertrigliceridemia, hyperbetalipoproteinemia (high LDL), hyperprebetalipoproteinemia (high VLDL), hyperchylomicronemia, hypolipoproteinemia, hypercholesterolemia, hyperlipoproteinemia and hypoalphalipoproteinemia (low HDL), as well as in the treatment of functional constipation or constipation-predominant irritable bowel syndrome. In particular, the solid formulation of the present invention can be used for the treatment or prevention of constipation in a warm-blooded animal including a human being. Here, the constipation mentioned above may be functional constipation or constipation-predominant irritable bowel syndrome.

### EXAMPLES

Hereinafter, the present invention is described in detail with reference to Examples and Comparative Examples. It should be understood that the present invention is not limited to these Examples.

### Reference Examples 1 to 10

1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiazepine (Elobixibat) and an additive shown in Table 1 were mixed in a weight ratio of 1:1. Thereby, the mixtures according to Reference Examples 2 to 10 were obtained. The mixture of each of Reference Examples 2 to 10, and only Elobixibat as Reference Example 1 was independently stored in an open state for 2 weeks under the conditions of 60° C. and 75% relative humidity. The material of Reference Example 1 and the mixtures according to Reference Examples 2 to 10 after storage were independently dispersed and dissolved in acetonitrile aqueous solution. The total amount of related substance(s) in the aforementioned aqueous solution was calculated as the sum of the areas in percentage for Elobixibat by the measurement in accordance with an HPLC method. In addition, the presence or absence of coloration of the mixtures of Reference Examples 1 to 7 after storage was visually observed. The results are shown in Table 1. “%” shown in Table 1 indicates percent by weight.

TABLE 1

| | | Amount of | Amount of | Total amount | Coloration after |
|---|---|---|---|---|---|
| | | plasticizer/ | plasticizer/ | of related | storage |
| | | Total weight | Weight of | substance(s) | Before storage → |
| | Additive | of formulation | drug substance | after storage | after storage |
| Reference | None | — | — | 0.6% | Not observed |
| Example 1 | | | | | White → white |
| Reference | Polyvinyl alcohol | 0% | 0% | 0.6% | Not observed |
| Example 2 | partial-saponification | | | | White → white |
| | product | | | | |
| Reference | Talc | 0% | 0% | 0.5% | Not observed |
| Example 3 | | | | | White → white |
| Reference | Titanium oxide | 0% | 0% | 0.6% | Not observed |
| Example 4 | | | | | White → white |
| Reference | Carnauba wax | 0% | 0% | 0.6% | Not observed |
| Example 5 | | | | | White → white |
| Reference | Macrogol 6000 | 50% | 50% | 2.2% | Observed |
| Example 6 | | | | | White → pale yellow |
| Reference | Macrogol 4000 | 50% | 50% | 2.0% | Observed |
| Example 7 | | | | | White → pale yellow |
| Reference | Triethyl citrate | 50% | 50% | 3.2% | — |
| Example 8 | | | | | |
| Reference | Triacetin | 50% | 50% | 6.5% | — |
| Example 9 | | | | | |
| Reference | Propylene glycol | 50% | 50% | 2.4% | — |
| Example 10 | | | | | |

As is clear from Table 1, in the mixture of Elobixibat with Macrogol (polyethylene glycol) 4000, Macrogol (polyethylene glycol) 6000, triethyl citrate, propylene glycol or triacetin, as a plasticizer, the amount of related substance(s) increased greatly. On the other hand, in the case of using only Elobixibat, or the mixture of Elobixibat with polyvinyl alcohol partial-saponification product, titanium oxide, talc, or carnauba wax, which was the ingredient other than the plasticizer, a great increase of the related substance(s) was not observed. Therefore, it can be seen that the great increase of the related substance (s) was caused by contacting Elobixibat as a drug substance with a relatively large amount of the specific plasticizer.

### Examples 1 to 6 and Comparative Examples 1 and 2

Crystalline cellulose (filler), D-mannitol (filler), Hypromellose (binder), croscarmellose sodium (disintegrant), light anhydrous silicic acid (fluidizer), magnesium stearate (lubricant), and 1,1-dioxo-3,3-dibutyl-5-phenyl-7-methylthio-8-(N—{(R)-1′-phenyl-1′-[N′-(carboxymethyl)carbamoyl]methyl}carbamoylmethoxy)-2,3,4,5-tetrahydro-1,5-benzothiazepine (Elobixibat) were formulated into tablets in accordance with a conventional method (mixing in a bag or mixing by means of a rotatory mixer, and tableting by means of a rotary tableting machine). Thereby, uncoated tablets (weight of uncoated tablet: 110 mg or 320 mg) containing 5% by weight of Elobixibat (drug substance) were obtained.

Hypromellose (coating agent) and Macrogol 6000 (plasticizer) were added to purified water, and mixed well until the mixture was dissolved. After the mixture was dissolved, titanium oxide (colorant) was added thereto, and mixed well to disperse it therein. The obtained mixture liquid was used as a film coating liquid. The compositions of the film coatings according to Examples 1 to 6 and Comparative Examples 1 and 2 are shown in Table 2.

The aforementioned film coating liquid was sprayed onto the aforementioned uncoated tablets by means of a pan-type coating machine. Thereby, film coating tablets were obtained.

The obtained film coating tablets in accordance with each of Examples 1 to 6 and Comparative Examples 1 and 2 were stored in an open state for 2 weeks under the conditions of 60° C. and 75% relative humidity. The tablets before or after storage were dissolved in an aqueous solution of acetonitrile. The total amount of related substance(s) in the aforementioned aqueous solution was calculated as the sum of the areas in percentage for Elobixibat by the measurement in accordance with an HPLC method. In addition, the presence or absence of coloration of the coating of the film coating tablets before or after storage was visually observed when the tablets were sealed in an aluminum bag as an airtight container, and then stored for 2 weeks under the conditions of 60° C. and 75% relative humidity. The results are shown in Table 2. “%” shown in Table 2 indicates percent by weight.

TABLE 2

| | | Example/Comparative Example | | | | | | | |
|---|---|---|---|---|---|---|---|---|---|
| | | | | | | | | Comparative | Comparative |
| | | Example 1 | Example 2 | Example 3 | Example 4 | Example 5 | Example 6 | Example 1 | Example 2 |
| Weight of uncoated | [mg/ | 110 | 110 | 110 | 320 | 320 | 110 | 110 | 320 |
| tablet | tablet] | | | | | | | | |
| Total weight | | 3.6 | 1.9 | 2.1 | 12.3 | 8.1 | 22.1 | 3.0 | 18.9 |
| of coating | | | | | | | | | |
| Hypromellose | [parts by | 83 | 71 | 63 | 71 | 63 | 83 | 48 | 48 |
| Macrogol 6000 | weight] | 8 | 21 | 31 | 21 | 31 | 8 | 48 | 48 |
| Titanium oxide | | 8 | 7 | 6 | 7 | 6 | 8 | 5 | 5 |
| Weight of Macrogol | [%] | 0.3% | 0.4% | 0.6% | 0.8% | 0.8% | 1.4% | 1.0% | 2.5% |
| 6000/Total | | | | | | | | | |
| weight of tablet | | | | | | | | | |
| Weight of Macrogol | | 6.0% | 8.3% | 13.4% | 17.6% | 16.9% | 36.8% | 21.9% | 55.2% |
| 6000/Weight of | | | | | | | | | |
| drug substance | | | | | | | | | |
| Weight of Macrogol | | 8% | 21% | 31% | 21% | 31% | 8.3% | 47.6% | 47.6% |
| 6000/weight of | | | | | | | | | |
| coating | | | | | | | | | |
| Total weight of | | 0.6% | 0.6% | 0.5% | 0.7% | 0.7% | 0.6% | 0.6% | 0.7% |
| related | | | | | | | | | |
| substance(s) | | | | | | | | | |
| before storage | | | | | | | | | |
| Total weight of | | 1.8% | 2.8% | 2.7% | 2.6% | 2.7% | 3.0% | 3.7% | 4.0% |
| related | | | | | | | | | |
| substance(s) | | | | | | | | | |
| after storage | | | | | | | | | |
| Coloration after | | Not observed | Not observed | Not observed | Not observed | Not observed | Not observed | Observed | Observed |
| storage *1 | | White → | White → | White → | White → | White → | White → | White → | White → |
| Before storage → | | white | white | white | white | white | white | pale yellow | slightly pale |
| after storage | | | | | | | | | yellow |

*1 The tablets were sealed in an aluminum bag as an airtight container, and then stored for 2 weeks under the conditions of 60° C. and 75% relative humidity.

As is clear from Table 2, when the drug substance contacts the plasticizer, in the case where the amount of the plasticizer is 0.9% by weight or less based on the total weight of the tablet, or alternatively, in the case where the amount of the plasticizer in the coating of the film coating tablet is 45% by weight or less based on the weight of the coating, the increasing of the total amount of the related substance(s) can be controlled, and coloration of the aforementioned tablets over time can be inhibited. It is preferable that the total weight of the related substance(s) be 3.0% by weight or less relative to the total weight of the tablet, as those according to Examples 1-6, in order to avoid pale yellow coloring of a tablet after storage.

1. A solid formulation characterized by comprising:
(A) a compound of formula (I) or (I′): 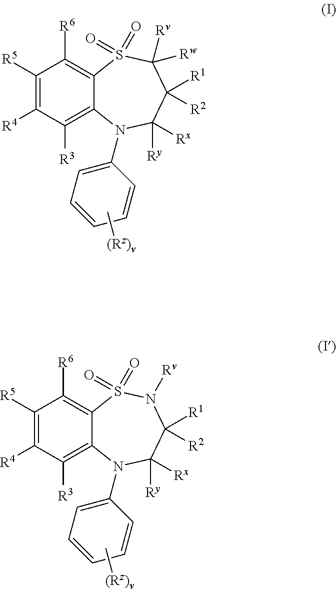
wherein:
R^{v} and R^{w} are independently selected from hydrogen or C₁₋₆ alkyl;
R¹ and R² are independently selected from C₁₋₆ alkyl;
R^{x} and R^{y} are independently selected from hydrogen or C₁₋₆ alkyl, or one of R^{x} and R^{y} is hydrogen or C₁₋₆ alkyl and the other is hydroxy or C₁₋₆ alkoxy;
R^{z} is selected from halo, nitro, cyano, hydroxy, amino, carboxy, carbamoyl, mercapto, sulphamoyl, C₁₋₆ alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl) carbamoyl, N,N—(C₁₋₆ alkyl)₂ carbamoyl, C₁₋₆ alkyl S(O)ₐ wherein a is 0 to 2, C₁₋₆ alkoxycarbonyl, C₁₋₆ alkoxycarbonylamino, ureido, N′—(C₁₋₆ alkyl)ureido, N—(C₁₋₆ alkyl)ureido, N′,N′—(C₁₋₆ alkyl)₂ureido, N′—(C₁₋₆ alkyl)-N—(C₁₋₆ alkyl)ureido, N′,N′—(C₁₋₆ alkyl)₂-N—(C₁₋₆ alkyl)ureido, N—(C₁₋₆ alkyl) sulphamoyl and N,N—(C₁₋₆ alkyl)₂ sulphamoyl;
v is 0 to 5;
one of R⁴ and R⁵ is a group of formula (IA): 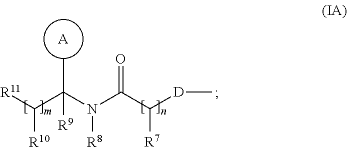
R³ and R⁶, and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆ alkyl and b is 0 to 2;
Ring A is aryl or heteroaryl; wherein Ring A is optionally substituted by one or more substituents selected from R¹⁷;
R⁷ is hydrogen, C₁₋₄ alkyl, carbocyclyl or heterocyclyl; wherein R⁷ is optionally substituted by one or more substituents selected from R¹⁸;
R⁸ is hydrogen or C₁₋₄ alkyl;
R⁹ is hydrogen or C₁₋₄ alkyl;
R¹⁰ is hydrogen, C₁₋₄ alkyl, carbocyclyl or heterocyclyl; wherein R¹⁰ is optionally substituted by one or more substituents selected from R⁹;
R¹¹ is carboxy, sulpho, sulphino, phosphono, tetrazolyl, —P(O)(OR^{c})(OR^{d}), —P(O)(OH)(OR^{c}), —P(O)(OH)(R^{d}) or —P(O)(OR^{c})(R^{d}) wherein R^{c} and R^{d} are independently selected from C₁₋₆ alkyl; or R¹¹ is a group of formula (IB): 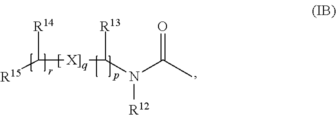
wherein:
X is —N(R^{q})—, —N(R^{q})C(O)—, —O—, or —S(O)ₐ—; wherein a is 0 to 2 and R^{q} is hydrogen or C₁₋₄ alkyl;
R¹² is hydrogen or C₁₋₄ alkyl;
R¹³ and R¹⁴ are independently selected from hydrogen, C₁₋₄ alkyl, carbocyclyl, heterocyclyl or R²³; wherein said C₁₋₄ alkyl, carbocyclyl or heterocyclyl may be independently optionally substituted by one or more substituents selected from R²⁰;
R¹⁵ is carboxy, sulpho, sulphino, phosphono, tetrazolyl, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆ alkyl; or R¹⁵ is a group of formula (IC): 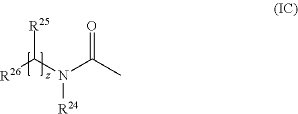
wherein:
R²⁴ is selected from hydrogen or C₁₋₄ alkyl;
R²⁵ is selected from hydrogen, C₁₋₄ alkyl, carbocyclyl, heterocyclyl or R²⁷; wherein said C₁₋₄ alkyl, carbocyclyl or heterocyclyl may be independently optionally substituted by one or more substituents selected from R²⁸;
R²⁶ is selected from carboxy, sulpho, sulphino, phosphono, tetrazolyl, —P(O)(OR^{g})(OR^{h}), —P(O)(OH)(OR^{g}), —P(O)(OH)(R^{g}) or —P(O)(OR^{g})(R^{h}) wherein R^{g} and R^{h} are independently selected from C₁₋₆ alkyl;
p is 1-3; wherein the meanings of R¹³ may be the same or different;
q is 0-1;
r is 0-3; wherein the meanings of R¹⁴ may be the same or different;
m is 0-2; wherein the meanings of R¹⁰ may be the same or different;
n is 1-3; wherein the meanings of R⁷ may be the same or different;
z is 0-3; wherein the meanings of R²⁵ may be the same or different;
R¹⁶, R¹⁷ and R¹⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl) amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R¹⁶, R¹⁷ and R¹⁸ may be independently optionally substituted on carbon by one or more R²¹;
R¹⁹, R²⁰, R²³, R²⁷ and R²⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆ alkyl; wherein R¹⁹, R²⁰, R²³, R²⁷ and R²⁸ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl, or a pharmaceutically acceptable salt, solvate, or solvate of such a salt; and
(B) at least one selected from the group consisting of polyethylene glycol, propylene glycol, glycerol, glyceryl triacetate, triethyl acetylcitrate, dibutyl sebacate, diethyl phthalate, castor oil, a copolymer of propylene oxide and ethylene oxide, triacetin, triethyl citrate, and a mixture thereof,
wherein
said ingredient (A) is isolated from said ingredient (B), or
in the case of said ingredient (A) not being isolated from said ingredient (B),
an amount of said ingredient (B) is 0.9% by weight or less based on the total weight of the solid formulation,
or
the solid formulation comprises
at least one core, and
at least one coating layer or capsule layer enclosing at least a part of said core,
where
said core comprises said ingredient (A),
said coating layer or capsule layer comprises said ingredient (B), and
an amount of said ingredient (B) in said coating layer or capsule layer is 45% by weight or less based on the total weight of said coating layer or capsule layer.

2. The solid formulation according to claim 1, wherein said solid formulation comprises:
at least one core, and
at least one coating layer or capsule layer enclosing at least a part of said core,
where
said core comprises said ingredient (A),
said coating layer or capsule layer comprises said ingredient (B), and
at least one isolation layer is provided between said core and said coating layer or capsule layer, or
in the case of said core contacting said coating layer or capsule layer,
an amount of said ingredient (B) in said coating layer or capsule layer is 0.9% by weight or less based on the total weight of the solid formulation,
or
an amount of said ingredient (B) in said coating layer or capsule layer is 45% by weight or less based on the total weight of said coating layer or capsule layer.

3. The solid formulation according to claim 2, wherein in the case of said core contacting said coating layer or capsule layer, an amount of said ingredient (B) in said coating layer or capsule layer ranges from 0.1 to less than 40% by weight based on the total weight of said coating layer or capsule layer.

4. The solid formulation according to claim 3, wherein said coating layer or capsule layer is present in a ratio ranging from 1 to 20% by weight based on the total weight of the solid formulation.

5. The solid formulation according to claim 1, wherein said coating layer or capsule layer further comprises at least one selected from the group consisting of a water-soluble polymer other than polyethylene glycol, a colorant, a lubricant, and wax.

6. The solid formulation according to claim 5, wherein said water-soluble polymer is hydroxypropylmethylcellulose.

7. The solid formulation according to claim 5, wherein said colorant is selected from the group consisting of titanium oxide, iron oxide, zinc oxide, tar pigment, and lake pigment.

8. The solid formulation according to claim 5, wherein said lubricant is talc.

9. The solid formulation according to claim 5, wherein said wax is carnauba wax.

10. The solid formulation according to claim 1, wherein said core comprises at least one additive selected from the group consisting of a filler, a disintegrant, a binder, a lubricant, and a fluidizer.

11. The solid formulation according to claim 1, which is a film coating tablet or a capsule.

12. The solid formulation according to claim 1, wherein an amount of said ingredient (B) ranges from 0.1 to 20% by weight based on the total weight of said ingredient (A).

13. The solid formulation according to claim 1, wherein said ingredient (A) is selected from the group consisting of:
1,1-dioxo-3,3-dibutyl-5-phenyl-7-methylthio-8-(N—{(R)-α-[N-(2-sulphoethyl)carbamoyl]-4-hydroxybenzyl} carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-(R)-hydroxypropyl)carbamoyl]-4-hydroxybenzoy}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxy-2-methylpropyl)carbamoyl]-4-hydroxybenzyl}carbamoylmethoxy)-2,3,4,5-tetrahydro-1,2,5-benzothiadiazepine;
1,1-dioxo-3,3-dibutyl-5-phenyl-7-methylthio-8-(N—{(R)-α-[N—((S)-1-carboxypropyl)carbamoyl]-4-hydroxybenzyl}carbamoylmethoxy)-2,3,4,5-tetrahydro-1,2,5-benzothiadiazepine; and
1,1-dioxo-3,3-dibutyl-5-phenyl-7-methylthio-8-(N—{(R)-1′-phenyl-1′-[N′-(carboxymethyl)carbamoyl]methyl} carbamoylmethoxy)-2,3,4,5-tetrahydro-1,5-benzothiazepine.

14. The solid formulation according to claim 1, wherein said ingredient (A) is Elobixibat.

15. The solid formulation according to claim 1, wherein an amount of said ingredient (A) ranges from 0.01 to 50% by weight based on the total weight of the solid formulation.

16. The solid formulation according to claim 1, wherein an amount of said ingredient (A) ranges from 1 to 20 mg.

17. The solid formulation according to claim 1, wherein said polyethylene glycol has an average molecular weight ranging from 200 to 20000.

18. The solid formulation according to claim 1, which is in the form of a tablet having a diameter ranging from 5 to 11 mm.

19. The solid formulation according to claim 1, which is intended for treating or preventing constipation in a warm-blooded animal including a human being.

20. The solid formulation according to claim 19, wherein the constipation is functional constipation or constipation-predominant irritable bowel syndrome.

21. A method for stabilizing the following ingredient (A) in a solid formulation comprising:
(A) a compound of formula (I) or (I′): 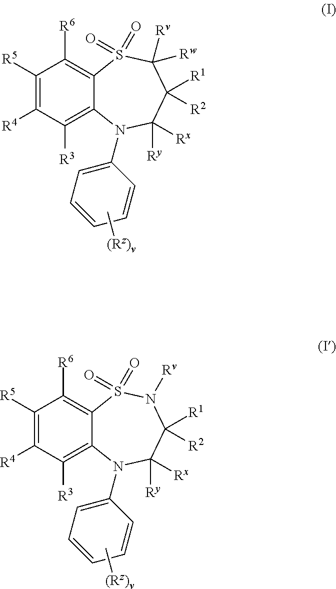
wherein:
R^{v} and R^{w} are independently selected from hydrogen or C₁₋₆ alkyl;
R¹ and R² are independently selected from C₁₋₆ alkyl;
R^{x} and R^{y} are independently selected from hydrogen or C₁₋₆ alkyl, or one of R^{x} and R^{y} is hydrogen or C₁₋₆ alkyl and the other is hydroxy or C₁₋₆ alkoxy;
R^{z} is selected from halo, nitro, cyano, hydroxy, amino, carboxy, carbamoyl, mercapto, sulphamoyl, C₁₋₆ alkyl, C₂₋₆ alkenyl, C₂₋₆ alkynyl, C₁₋₆ alkoxy, C₁₋₆ alkanoyl, C₁₋₆ alkanoyloxy, N—(C₁₋₆ alkyl)amino, N,N—(C₁₋₆ alkyl)₂ amino, C₁₋₆ alkanoylamino, N—(C₁₋₆ alkyl)carbamoyl, N,N—(C₁₋₆ alkyl)₂ carbamoyl, C₁₋₆ alkyl S(O)ₐ wherein a is 0 to 2, C₁₋₆ alkoxycarbonyl, C₁₋₆ alkoxycarbonylamino, ureido, N′—(C₁₋₆ alkyl)ureido, N—(C₁₋₆ alkyl)ureido, N′,N′—(C₁₋₆ alkyl)₂ ureido, N′—(C₁₋₆ alkyl)-N—(C₁₋₆ alkyl)ureido, N′,N′—(C₁₋₆ alkyl)₂-N—(C₁₋₆ alkyl)ureido, N—(C₁₋₆ alkyl)sulphamoyl and N,N—(C₁₋₆ alkyl)₂ sulphamoyl;
v is 0 to 5;
one of R⁴ and R⁵ is a group of formula (IA): 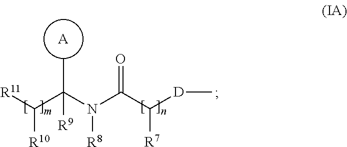
R³ and R⁶ and the other of R⁴ and R⁵ are independently selected from hydrogen, halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R³ and R⁶ and the other of R⁴ and R⁵ may be optionally substituted on carbon by one or more R¹⁶;
D is —O—, —N(R^{a})—, —S(O)_{b}— or —CH(R^{a})—; wherein R^{a} is hydrogen or C₁₋₆ alkyl and b is 0 to 2;
Ring A is aryl or heteroaryl; wherein Ring A is optionally substituted by one or more substituents selected from R¹⁷;
R⁷ is hydrogen, C₁₋₄ alkyl, carbocyclyl or heterocyclyl; wherein R⁷ is optionally substituted by one or more substituents selected from R¹⁸;
R⁸ is hydrogen or C₁₋₄ alkyl;
R⁹ is hydrogen or C₁₋₄ alkyl;
R¹⁰ is hydrogen, C₁₋₄ alkyl, carbocyclyl or heterocyclyl; wherein R¹⁰ is optionally substituted by one or more substituents selected from R¹⁹;
R¹¹ is carboxy, sulpho, sulphino, phosphono, tetrazolyl, —P(O)(OR^{c})(OR^{d}), —P(O)(OH)(OR^{c}), —P(O)(OH)(R^{d}) or —P(O)(OR)(R^{d}) wherein R^{c} and R^{d} are independently selected from C₁₋₆ alkyl; or R¹ is a group of formula (IB): 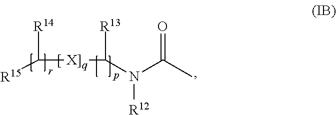
wherein:
X is —N(R^{q})—, —N(R^{q})C(O)—, —O—, or —S(O)ₐ—; wherein a is 0 to 2 and R^{q} is hydrogen or C₁₋₄ alkyl;
R¹² is hydrogen or C₁₋₄ alkyl;
R¹³ and R¹⁴ are independently selected from hydrogen, C₁₋₄ alkyl, carbocyclyl, heterocyclyl or R²³; wherein said C₁₋₄ alkyl, carbocyclyl or heterocyclyl may be independently optionally substituted by one or more substituents selected from R²⁰;
R¹⁵ is carboxy, sulpho, sulphino, phosphono, tetrazolyl, —P(O)(OR^{e})(OR^{f}), —P(O)(OH)(OR^{e}), —P(O)(OH)(R^{e}) or —P(O)(OR^{e})(R^{f}) wherein R^{e} and R^{f} are independently selected from C₁₋₆ alkyl; or R¹⁵ is a group of formula (IC): 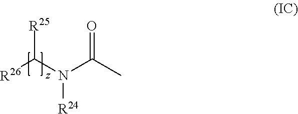
wherein:
R²⁴ is selected from hydrogen or C₁₋₄ alkyl;
R²⁵ is selected from hydrogen, C₁₋₄ alkyl, carbocyclyl, heterocyclyl or R²⁷; wherein said C₁₋₄ alkyl, carbocyclyl or heterocyclyl may be independently optionally substituted by one or more substituents selected from R²⁸;
R²⁶ is selected from carboxy, sulpho, sulphino, phosphono, tetrazolyl, —P(O)(OR^{g})(OR^{h}), —P(O)(OH)(OR^{g}), —P(O)(OH)(R^{g}) or —P(O)(OR^{g})(R^{h}) wherein R^{g} and
R^{h} are independently selected from C₁₋₆ alkyl;
p is 1-3; wherein the meanings of R¹³ may be the same or different;
q is 0-1;
r is 0-3; wherein the meanings of R¹⁴ may be the same or different;
m is 0-2; wherein the meanings of R¹⁰ may be the same or different;
n is 1-3; wherein the meanings of R⁷ may be the same or different;
z is 0-3; wherein the meanings of R²⁵ may be the same or different;
R¹⁶, R¹⁷ and R¹⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl and N,N—(C₁₋₄ alkyl)₂ sulphamoyl; wherein R¹⁶, R¹⁷ and R¹⁸ may be independently optionally substituted on carbon by one or more R²¹;
R¹⁹, R²⁰, R²³, R²⁷ and R²⁸ are independently selected from halo, nitro, cyano, hydroxy, amino, carboxy, carbamoyl, mercapto, sulphamoyl, C₁₋₄ alkyl, C₂₋₄ alkenyl, C₂₋₄ alkynyl, C₁₋₄ alkoxy, C₁₋₄ alkanoyl, C₁₋₄ alkanoyloxy, N—(C₁₋₄ alkyl)amino, N,N—(C₁₋₄ alkyl)₂ amino, C₁₋₄ alkanoylamino, N—(C₁₋₄ alkyl)carbamoyl, N,N—(C₁₋₄ alkyl)₂ carbamoyl, C₁₋₄ alkyl S(O)ₐ wherein a is 0 to 2, C₁₋₄ alkoxycarbonyl, N—(C₁₋₄ alkyl)sulphamoyl, N,N—(C₁₋₄ alkyl)₂ sulphamoyl, carbocyclyl, heterocyclyl, sulpho, sulphino, amidino, phosphono, —P(O)(OR^{a})(OR^{b}), —P(O)(OH)(OR^{a}), —P(O)(OH)(R^{a}) or —P(O)(OR^{a})(R^{b}), wherein R^{a} and R^{b} are independently selected from C₁₋₆ alkyl; wherein R¹⁹, R²⁰, R²³, R²⁷ and R²⁸ may be independently optionally substituted on carbon by one or more R²²;
R²¹ and R²² are independently selected from halo, hydroxy, cyano, carbamoyl, ureido, amino, nitro, carboxy, carbamoyl, mercapto, sulphamoyl, trifluoromethyl, trifluoromethoxy, methyl, ethyl, methoxy, ethoxy, vinyl, allyl, ethynyl, methoxycarbonyl, formyl, acetyl, formamido, acetylamino, acetoxy, methylamino, dimethylamino, N-methylcarbamoyl, N,N-dimethylcarbamoyl, methylthio, methylsulphinyl, mesyl, N-methylsulphamoyl and N,N-dimethylsulphamoyl, or a pharmaceutically acceptable salt, solvate, or solvate of such a salt; and
(B) at least one selected from the group consisting of polyethylene glycol, propylene glycol, glycerol, glyceryl triacetate, triethyl acetylcitrate, dibutyl sebacate, diethyl phthalate, castor oil, a copolymer of propylene oxide and ethylene oxide, triacetin, triethyl citrate, and a mixture thereof,
the method being characterized by comprising:
isolating said ingredient (A) from said ingredient (B); or
in the case of said ingredient (A) not being isolated from said ingredient (B),
controlling an amount of said ingredient (B) to 0.9% by weight or less based on the total weight of the solid formulation,
or
providing the solid formulation with a form comprising at least one core and at least one coating layer or capsule layer enclosing at least a part of said core,
mixing said ingredient (A) into said core,
mixing said ingredient (B) into said coating layer or capsule layer, and
controlling an amount of said ingredient (B) in said coating layer or capsule layer to 45% by weight or less based on the total weight of said coating layer or capsule layer.
